(19) United States
(12) Patent Application Publication
(10) Pub. No.: US 2007/0185132 A1
(43) Pub. Date: Aug. 9, 2007

(54) Cyclopropyl group substituted oxazolidinone antibiotics and derivatives thereo

(76) Inventor: Yasumichi Fukuda, Tochigi (JP)

Correspondence Address: MCDERMOTT WILL & EMERY LLP, 600 13TH STREET, N.W., WASHINGTON, DC, 20005-3096, US

(21) Appl. No.: 10/559,869

(22) PCT Filed: Jun. 29, 2004
(86) PCT No.: PCT/US04/20737

§ 371 Date: Mar. 19, 2007

Related U.S. Application Data
(60) Provisional application No. 60/483,904, filed on Jul. 2, 2003.
(60) Provisional application No. 60/546,980, filed on Feb. 24, 2004.

(51) Int. Cl. A61K 31/506; A61K 31/4747; A61K 31/4439; C07D 413/14; C07D 413/02

(52) U.S. Cl. 514/256; 514/340; 546/271.4; 514/278; 546/15; 544/331

(57) ABSTRACT

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.
The compounds are represented by structural formula I: 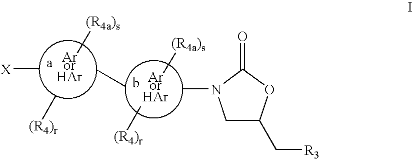 its enantiomer, diastereomer, or pharmaceutically acceptable salt or ester thereof.

### CROSS REFERENCE TO RELATED APPLICATIONS

This application claims priority to Provisional Application 60/483,904 FILED Jul. 2, 2003 and Provisional Application 60/546,980 filed Feb. 24, 2004, which are hereby incorporated herein by reference in their entirety.

### BACKGROUND OF THE INVENTION

Oxazolidinones represent the first new class of antibacterials to be developed since the quinolones. The oxazolidinones are synthetic antibacterial compounds that are orally or intravenously active against problematic multidrug resistant Gram positive organisms and are not cross-resistant with other antibiotics. See Riedl et al, Recent Developments with Oxazolidinone Antibiotics, Exp. Opin. Ther. Patents (1999) 9(5), Ford et al., Oxazolidinones: New Antibacterial Agents, Trends in Microbiology 196 Vol. 5, No. 5, May 1997 and WO 96/35691. See also WO 03/063862, WO 01/81350, WO 01/94342, WO 03/072553, EP 0352781 and U.S. Pat. Nos. 5,565,571 and 4,053,593.

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.

### SUMMARY OF THE INVENTION

The present invention relates to compounds of formula I: 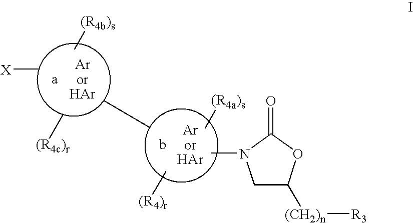
its enantiomer, diastereomer, or pharmaceutically acceptable salt, hydrate or prodrug thereof wherein:
R₁ and R₂ independently represent
i) hydrogen,
ii) (CH₂)ₙNR₅R₆,
iii) CR₇R₈R₉, C(R)₂OR₁₄, CH₂NHR₁₄,
iv) C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄,
v) (C₁₋₆alkyl)CN, CN, CH═C(R)₂, (CH₂)ₚOH, C(═O)CHR₁₃, C(═NR₁₃)R₁₃, NR₁₀C(═X₁)R₁₃; or
vi) C₅₋₁₀ heterocycle optionally substituted with 1-3 groups of R₇, which may be attached through either a carbon or a heteroatom;
R₁ₐ represents (CH₂)ₙNR₅R₆, CR₇R₈R₉, C(R)₂OR₁₄, CH₂NHR₁₄, C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄, (C₁₋₆alkyl)CN, CN, CH═C(R)₂, (CH₂)ₚOH, C(═O)CHR₁₃, C(═NR₁₃)R₁₃, NR₁₀C(═X₁)R₁₃; or C₅₋₁₀ heterocycle optionally substituted with 1-3 groups of R₇, which may be attached through either a carbon or a heteroatom;
X is selected from the group consisting of, 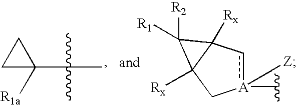
   Z represents (O)ₙ, H, OH, or halogen;
A represents C (when --- is present provided Z=(O)ₙ and n=0), C (when --- is not present provided Z is H, OH or halogen), or N (when --- is not present and Z=(O)ₙ and n=1);
--- represents a bond; 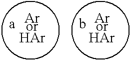
   represents aryl or heteroaryl, heterocycle, heterocyclyl or heterocyclic, provided that in the case of a heteroaryl, heterocycle, heterocyclyl or heterocyclic, the cyclopropyl is not attached to a nitrogen atom on the ring;
Rₓ represents hydrogen or C₁₋₆ alkyl;
R₃ represent

i) NR₁₃(C═X₂)R₁₂,

ii) NR₁₃(C═X₁)R₁₂,

iii) NR₁₃SO₂R₁₄,

iv) N(R₁₃)heteroaryl,

v) NR₁₃(CHR₁₃)₀₋₄aryl,

vi) NR₁₃(CHR₁₃)₀₋₄heteroaryl,

vii) S(CHR₁₃)₀₋₄aryl,

viii) S(CHR₁₃)₀₋₄heteroaryl,

ix) O(CHR₁₃)₀₋₄aryl,

x) O(CHR₁₃)₀₋₄heteroaryl,

xi) NOH(C═X₁)R₁₂,

xii) —OC═N(OCOaryl) C₁₋₆ alkyl

xiii) —OC═N(OH) C₁₋₆ alkyl

xiv) C₅₋₁₀ heteroaryl which may be attached through either a carbon or a heteroatom; said aryl and heteroaryl optionally substituted with 1-3 groups of R₇,
R₄, R₄ₐ, R_{4b}, and R_{4c} independently represent
i) hydrogen,
ii) halogen,
iii) C₁₋₆ alkoxy, or
iv) C₁₋₆ alkyl
r and s independently are 1-3, with the provision that when (R₄ₐ)ₛ and (R₄)ᵣ or (R_{4b}) and (R_{4c})ₛ are attached to an Ar or HAr ring the sum of r and s is less than or equal to 4;
R₅ and R₆ independently represent
i) hydrogen,
ii) C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;
iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
iv) C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl;
v) arylsulfonyl optionally substituted with 1-3 of halogen, C₁₋₆ alkoxy, OH or C₁₋₆ alkyl;
vi) C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl;
vii) aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl
viii) five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
ix) C₃₋₆ cycloalkylcarbonyl optionally substituted with 1-3 groups of halogen, OH, C₁₋₆ alkoxy or CN;
x) benzoyl optionally substituted with 1-3 groups of halogen, OH, C₁₋₆ alkoxy, C₁₋₆ alkyl, CF₃, C₁₋₆ alkanoyl, amino or C₁₋₆ acylamino;
xi) pyrrolylcarbonyl optionally substituted with 1-3 of C₁₋₆ alkyl;
xii) C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or
R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
i) hydrogen, halogen, CN, CO₂R, CON(R)₂, CHO, (CH₂)₀₋₃NHAc, C(═NOR), OH, C₁₋₆ alkoxy, C₁₋₆ alkyl, alkenyl, hydroxy C₁₋₆ alkyl, (CH₂)₁₋₃NHC(O)C₁₋₆ alkyl, (CH₂)₀₋₃N(C₁₋₆ alkyl)₂
ii) (CH₂)ₙamino, (CH₂)ₙC1-6 alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
i) H, CN,
ii) C₁₋₆ alkyl optionally substituted with 1-3 halogen, CN, OH, C₁₋₆ alkoxy, C₁₋₆ acyloxy, or amino,
iii) phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
X₂ represents O, S, NH or NSO₂R₁₄;
R₁₀ represents hydrogen, C₁₋₆ alkyl or CO₂R₁₅;
R₁₂ represents hydrogen, C₁₋₆ alkyl, NH₂, OR, CHF₂, CHCl₂, CR₂Cl, (CH₂)ₙSR, (CH₂)ₙCN, (CH₂)ₙSO₂R, (CH₂)ₙS(O)R, C₁₋₆ alkylamino, C₅₋₁₀ heteroaryl or C₁₋₆ dialkylamino, where said alkyl may be substituted with 1-3 groups of halo, CN, OH or C₁₋₆ alkoxy, said heteroaryl optionally substituted with 1-3 groups of R₇;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, —OCOaryl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen or C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
p represents 0-2 and
m, n, and q represents 0-1.

Another aspect of the invention is concerned with the use of the novel antibiotic compositions in the treatment of bacterial infections.

### DETAILED DESCRIPTION OF THE INVENTION

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994, in particular pages 1119-1190).

When any variable (e.g. aryl, heterocycle, R₅, R₆ etc.) occurs more than once, its definition on each occurrence is independent at every other occurrence. Also combinations of substituents/or variables are permissible only if such combinations result in stable compounds.

The term “alkyl” refers to a monovalent alkane (hydrocarbon) derived radical containing from 1 to 15 carbon atoms unless otherwise defined. It may be straight or branched. Preferred alkyl groups include lower alkyls which have from 1 to 6 carbon atoms such as methyl, ethyl, propyl, isopropyl, butyl and t-butyl. When substituted, alkyl groups may be substituted with up to 3 substituent groups, selected from the groups as herein defined, at any available point of attachment. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused. Preferred cycloalkyl groups are cyclopropyl, cyclobutyl, cyclopentyl and cyclohexyl. When substituted, cycloalkyl groups may be substituted with up to 3 substituents which are defined herein by the definition of alkyl.

Alkanoyl refers to a group derived from an aliphatic carboxylic acid of 2 to 4 carbon atoms. Examples are acetyl, propionyl, butyryl and the like.

The term “alkoxy” refers to those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like. 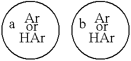
refers to aryl or heteroaryl, heterocycle, Het, heterocyclyl or heterocyclic as described immediately below.

Aryl refers to any stable monocyclic or bicyclic carbon ring of up to 7 atoms in each ring, wherein at least one ring is aromatic. Examples of such aryl elements include phenyl, napthyl, tetrahydronaphthyl, indanyl, indanonyl, biphenyl, tetralilnyl, tetralonyl, fluorenonyl, phenanthryl, anthryl, acenaphthyl, and the like substituted phenyl and the like. Aryl groups may likewise be substituted as defined. Preferred substituted aryls include phenyl and naphthyl.

The term heterocycle, heteroaryl, Het, heterocyclyl or heterocyclic, as used herein except where noted, represents a stable 5- to 7-membered mono- or bicyclic or stable 8- to 11-membered bicyclic heterocyclic ring system, any ring of which may be saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized (in which case it is properly balanced by a counterion), and including any bicyclic group in which any of the above-defined heterocyclic rings is fused to a benzene ring. The heterocyclic ring may be attached at any heteroatom or carbon atom, which results in the creation of a stable structure. The term heterocycle or heterocyclic includes heteroaryl moieties. “Heterocycle” or “heterocyclyl” therefore includes the above mentioned heteroaryls, as well as dihydro and tetrahydro analogs thereof. The heterocycle, heteroaryl, Het or heterocyclic may be substituted with 1-3 groups of R₇. Examples of such heterocyclic elements include, but are not limited to the following: piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyrimidonyl, pyridinonyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thiophenyl, imidazopyridinyl, triazolyl, tetrazolyl, triazinyl, thienyl, benzothienyl, thiamorpholinyl sulfoxide, thiamorpholinyl sulfone, naphthpyridinyl, dihydropyrimidinyl, dihydropyrrolyl, dihydroquinolinyl, dihydrotetrazolyl, dihydrotriazolyl, dihydrothienyl, dihydrooxazolyl, dihydrobenzothiophenyl, dihydrofuranyl, benzothiazolyl, benzothienyl, benzoimidazolyl, benzopyranyl, benzothiofuranyl, carbolinyl, chromanyl, cinnolinyl, benzopyrazolyl, benzodioxolyl and oxadiazolyl. Additional examples of heteroaryls are illustrated by formulas a, b, c and d: 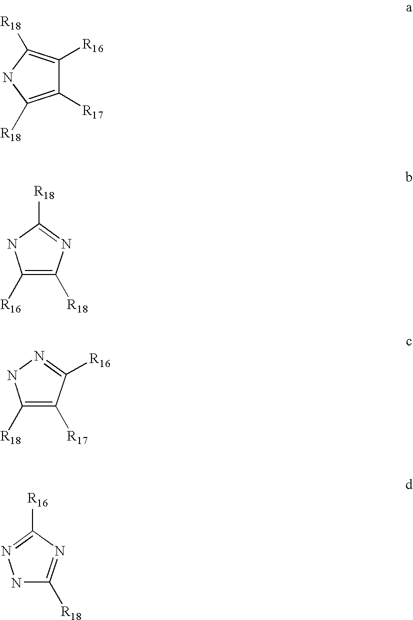
wherein R₁₆ and R₁₇ are independently selected from hydrogen, halogen, C₁₋₆ alkyl, C₂₋₄ alkanoyl, C₁₋₆ alkoxy; and R₁₈ represents hydrogen, C₁₋₆ alkyl, C₂₋₄ alkanoyl, C₁₋₆ alkoxycarbonyl and carbamoyl.

The term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl.

The terms “quaternary nitrogen” and “positive charge” refer to tetravalent, positively charged nitrogen atoms (balanced as needed by a counterion known in the art) including, e.g., the positively charged nitrogen in a tetraalkylammonium group (e.g. tetramethylammonium), heteroarylium, (e.g., N-methyl-pyridinium), basic nitrogens which are protonated at physiological pH, and the like. Cationic groups thus encompass positively charged nitrogen-containing groups, as well as basic nitrogens which are protonated at physiologic pH.

The term “heteroatom” means O, S or N, selected on an independent basis.

The term “prodrug” refers to compounds which are drug precursors which, following administration and absorption, release the drug in vivo via some metabolic process. Exemplary prodrugs include acyl amides of the amino compounds of this inventon such as amides of alkanoic(C₁₋₆)acids, amides of aryl acids (e.g., benzoic acid) and alkane(C₁₋₆)dioic acids.

Halogen and “halo” refer to bromine, chlorine, fluorine and iodine.

When a group is termed “substituted”, unless otherwise indicated, this means that the group contains from 1 to 3 substituents thereon.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

Examples of suitable hydroxyl and amino protecting groups are: trimethylsilyl, triethylsilyl, o-nitrobenzyloxycarbonyl, p-nitrobenzyloxycarbonyl, t-butyldiphenylsilyl, t-butyldimethylsilyl, benzyloxycarbonyl, t-butyloxycarbonyl, 2,2,2-trichloroethyloxycarbonyl, allyloxycarbonyl and the like. Examples of suitable carboxyl protecting groups are benzhydryl, o-nitrobenzyl, p-nitrobenzyl, 2-naphthylmethyl, allyl, 2-chloroallyl, benzyl, 2,2,2-trichloroethyl, trimethylsilyl, t-butyldimethylsilyl, t-butldiphenylsilyl, 2-(trimethylsilyl)ethyl, phenacyl, p-methoxybenzyl, acetonyl, p-methoxyphenyl, 4-pyridylmethyl, t-butyl and the like.

The cyclopropyl containing oxazolidinone compounds of the present invention are useful per se and in their pharmaceutically acceptable salt and ester forms for the treatment of bacterial infections in animal and human subjects. The term “pharmaceutically acceptable ester, salt or hydrate,” refers to those salts, esters and hydrated forms of the compounds of the present invention which would be apparent to the pharmaceutical chemist. i.e., those which are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug. Conveniently, pharmaceutical compositions may be prepared from the active ingredients in combination with pharmaceutically acceptable carriers. Thus, the present invention is also concerned with pharmaceutical compositions and methods of treating bacterial infections utilizing as an active ingredient the novel cyclopropyl containing oxazolidinone compounds.

The pharmaceutically acceptable salts referred to above also include acid addition salts. Thus, when the Formula I compounds are basic, salts may be prepared from pharmaceutically acceptable non-toxic acids, including inorganic or organic acids. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, isethionic, lactate, maleate, mandelic, malic, maleic, methanesulfonate, mucic, 2-naphthalenesulfonate, nicotinate, nitric oxalate, pamoate, pectinate, persulfate, 3-phenylpropionate, picrate, pivalate, propionate, phosphate, pantothenic, pamoic, sulfate, succinate, tartrate, thiocyanate, tosylate and undecanoate.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium zinc and the like. Particularly preferred are the ammonium, calcium, magnesium, potassium and sodium salts. Salts derived from pharmaceutically acceptable inorganic non-toxic bases include salts of primary, secondary and teritary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The pharmaceutically acceptable esters are such as would be readily apparent to a medicinal chemist, and include those which are hydrolyzed under physiological conditions, such as “biolabile esters”, pivaloyloxymethyl, acetoxymethyl, phthalidyl, indanyl and methoxymethyl, and others.

Biolabile esters are biologically hydrolizable, and may be suitable for oral administration, due to good absorption through the stomach or intenstinal mucosa, resistance to gastric acid degrada-tion and other factors. Examples of biolabile esters include compounds.

An embodiment of this invention is realized when R₁ and R₂ independently represent H, NR₅R₆, CN, OH, C(R)₂OR₁₄, NHC(═X1)N(R₁₃)₂, C(═NOH)N(R₁₃)₂, NR₁₀C(═X₁)R₁₃ or CR₇R₈R₉ and all other variables are as described herein.

Another embodiment of this invention is realized when R₁ₐ represents NR₅R₆, CN, OH, C(R)₂OR₁₄, NHC(═X1)N(R₁₃)₂, C(═NOH)N(R₁₃)₂, NR₁₀C(═X₁)R₁₃ or CR₇R₈R₉ and all other variables are as described herein.

Another embodiment of this invention is realized when 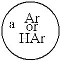
is phenyl, pyridine, pyrimidine, or piperidine and all other variables are as described herein.

Another embodiment of this invention is realized when 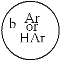
is phenyl, pyridine, pyrimidine, or piperidine and all other variables are as described herein.

Another embodiment of this invention is realized when one of R₁ and R₂ is H and the other is NR₅R₆ and all other variables are as described herein.

Another embodiment of this invention is realized when one of R₁ and R₂ is H and the other is CN and all other variables are as described herein.

Another embodiment of this invention is realized when R₁ₐ CN or NR₅R₆ and all other variables are as described herein.

Another embodiment of this invention is realized when one of R₁ and R₂ is H and the other is NR₁₀C(═X₁)R₁₃ and all other variables are as described herein.

An embodiment of this invention is realized when X is 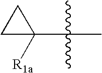
and all other variables are as described herein.

An embodiment of this invention is realized when X is 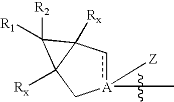
all other variables are as described herein. A subembodiment of this invention is realized when A is C, --- is present and Z=(O)ₙ where n=0, and all other variables are as described herein. Another sub-embodiment of this invention is realized when A is N and --- is not present and Z=(O)ₙ where n=1 and all other variables are as described herein. Still another sub-embodiment of this invention is realized when A is C, --- is not present and Z=H, OH or halogen where n=1 and all other variables are as described herein.

Another embodiment of this invention is realized when R₃ is NR(C═X₁)R₁₂, C₅₋₁₀ heteroaryl, NH(CH₂)₀₋₄aryl, NH(CH₂)₀₋₄heteroaryl, said aryl and heteroaryl optionally substituted with 1-3 groups of Rₐ and all other variables are as described herein.

Another embodiment of this invention is realized when R₃ is a C₅₋₁₀ heteroaryl represented by 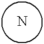
which represents an optionally substituted a romatic heterocyclic group containing 1 to 4 nitrogen atoms and at least one double bond, and which is connected through a bond on any nitrogen. E xemplary groups are 1,2,3-triazole, 1,2,4-triazole, 1,2,5-triazole, tetrazole, pyrazole, and imidazole, any of which may contain 1 to 3 substitutents selected from R₇.

Still another embodiment of this invention is realized when R₅ and R₆ independently are:
i) H,
ii) C₁₋₆ alkyl optionally substituted with 1-3 groups of halogen, CN, OH, C₁₋₆ alkoxy, amino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethyenyloxy, or ethynyl, said phenyl and pyridine optionally substituted with 1-3 halogen, CN, OH, CF₃, C₁₋₆ alkyl or C₁₋₆ alkoxy;
iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl; or
iv) benzoyl optionally substituted with 1-3 groups of halogen, OH, C₁₋₆ alkoxy, C₁₋₆ alkyl, CF3, C₁₋₆ alkanoyl, amino or C₁₋₆ acylamino and all other variables are as described herein.

Yet another embodiment of this invention is realized when X₁ represents O and all other variables are as described herein.

A preferred embodiment of this invention is realized when the structural formula is II: 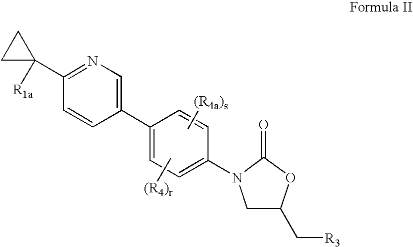
wherein R₁ₐ, R₄, R₄ₐ, and R₃ are as described herein.

Another preferred embodiment of this invention is realized when R₁ₐ is CN or NR₅R₆.

A preferred embodiment of this invention is realized when the structural formula is III: 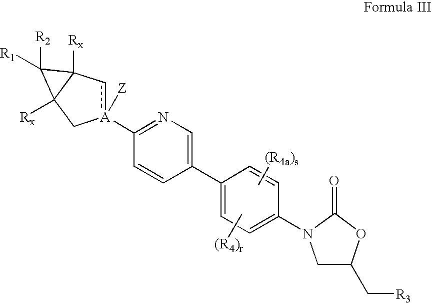
wherein Z, R₁, R₂, Rₓ, R₄, R₄ₐ, A and R₃ are as described herein.

### Preferred Compounds of this Invention are

N-[5(S)-3-[4-[2-(1-cyanocyclopropan-1-yl)pyridin-5-yl]phenyl]-2-oxooxazolidin-5-ylmethyl]acetamide,
N-[5(S)-3-[4-[5-(1-cyanocyclopropan-1-yl)pyridin-2-yl]phenyl]-2-oxooxazolidin-5-ylmethyl]acetamide,
N-[5(S)-3-[4-[2-(1-cyanocyclopropan-1-yl)pyridin-5-yl]-3-fluorophenyl]-2-oxooxazolidin-5-ylmethyl]acetamide,
N-[5(S)-3-[4-[2-(1-(t-butoxycarbonyl)aminocyclopropan-1-yl)pyridin-5-yl]phenyl]-2-oxooxazolidin-5-ylmethyl]acetamide,
N-[5(S)-3-[4-[2-(1-aminocyclopropan-1-yl)pyridin-5-yl]phenyl]-2-oxooxazolidin-5-ylmethyl]acetamide,
N-[5(S)-3-[4-[2-(1-(t-butoxycarbonyl)aminocyclopropan-1-yl)pyridin-5-yl]-3-fluorophenyl]-2-oxooxazolidin-5-ylmethyl]acetamide,
N-[5(S)-3-[4-[2-(1-aminocyclopropan-1-yl)pyridin-5-yl]-3-fluorophenyl]-2-oxooxazolidin-5-ylmethyl]acetamide,
N-[5(S)-3-[4-[2-(1-(dimethylamino)methylcyclopropan-1-yl)pyridin-5-yl]-3-fluorophenyl]-2-oxooxazolidin-5-ylmethyl]acetamide,
N-[5(S)-3-[4-[2-(1-(dimethylamino)methylcyclopropan-1-yl)pyridin-5-yl]-3-fluorophenyl]-2-oxooxazolidin-5-ylmethyl]acetamide,
N-[5(S)-3-[4-[2-(1-t-butoxycarbonylcyclopropan-1-yl)pyridin-5-yl]-3-fluorophenyl]-2-oxooxazolidin-5-ylmethyl]acetamide,
N-[5(S)-3-[4-[2-(1-hydroxymethylcyclopropan-1-yl)pyridin-5-yl]-3-fluorophenyl]-2-oxooxazolidin-5-ylmethyl]acetamide,
N-[5(S)-3-[4-[2-(1-hydroxycarbonylcyclopropan-1-yl)pyridin-5-yl]-3-fluorophenyl]-2-oxooxazolidin-5-ylmethyl]acetamide,
N-[5(S)-3-[4-[2-(1-(t-butoxycarbonyl)aminocyclopropan-1-yl)pyridin-5-yl]-3,5-difluorophenyl]-2-oxooxazolidin-5-ylmethyl]acetamide,
N-[5(S)-3-[4-[2-(1-aminocyclopropan-1-yl)pyridin-5-yl]-3,5-difluorophenyl]-2-oxooxazolidin-5-ylmethyl]acetamide,
N-[5(S)-3-[4-[2-(1-aminomethylcyclopropan-1-yl)pyridin-5-yl]-3-fluorophenyl]-2-oxooxazolidin-5-ylmethyl]acetamide,
1-[5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]-1,2,3-triazole,
1-[5(R)-3-[4-[2-(1-aminocyclopropan-1-yl)pyridin-5-yl]-3-fluorophenyl]-2-oxooxazolidin-5-ylmethyl]-1,2,3-triazole,
1-[5(R)-3-[4-[2-(1-aminocyclopropan-1-yl)pyridin-5-yl]phenyl]-2-oxooxazolidin-5-ylmethyl]-1,2,3-triazole,
1-[5(R)-3-[4-[2-(1-cyanocyclopropan-1-yl)pyridin-5-yl]phenyl]-2-oxooxazolidin-5-ylmethyl]-1,2,3-triazole,
1-[5(R)-3-[4-[2-(1-cyanocyclopropan-1-yl)pyridin-5-yl]-3-fluorophenyl]-2-oxooxazolidin-5-ylmethyl]-1,2,3-triazole,
N-[5(R)-3-[4-[2-(1-cyanocyclopropan-1-yl)pyridin-5-yl]-3,5-difluorophenyl]-2-oxooxazolidin-5-ylmethyl]acetamide,
5(R)-3-[4-[2-(1-cyanocyclopropan-1-yl)pyridin-5-yl]phenyl]-5-[(isoxazol-3-yl)oxy]methyloxazolidin-2-one,
5(R)-3-[4-[2-(1-cyanocyclopropan-1-yl)pyridin-5-yl]phenyl]-5-[N-(t-butoxycarbonyl)-N-(isoxazol-3-yl)]aminomethyloxazolidin-2-one,
5(R)-3-[4-[2-(1-cyanocyclopropan-1-yl)pyridin-5-yl]phenyl]-5-[N-(isoxazol-3-yl)]aminomethyloxazolidin-2-one,
5(R)-3-[4-[2-(1-t-butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-t-aminocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-t-butoxycarbonylaminocyclopropan-1-yl)pyridin-5-yl]phenyl]-5-[(isoxazol-3-yl)oxy]methyloxazolidin-2-one,
5(R)-3-[4-[2-(1-aminocyclopropan-1-yl)pyridin-5-yl]phenyl]-5-[(isoxazol-3-yl)oxy]methyloxazolidin-2-one,
5(R)-3-[4-[2-(1-cyanocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-cyanocyclopropan-1-yl)pyridin-5-yl]-3-fluorophenyl]-5-[N-(isoxazol-3-yl)]aminomethyloxazolidin-2-one,
5(R)-3-[4-[2-(1-cyanocyclopropan-1-yl)pyridin-5-yl]-3-fluorophenyl]-5-[(isoxazol-3-yl)oxy]methyloxazolidin-2-one,
1-[5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]-1,2,3-triazole,
1-[5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]-1,2,3-triazole,
5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]acetamide,
5(R)-3-[4-[2-[(1α,5α,6α)-6-amino-3-azabicyclo[3.1.0]hexan-3-yl]pyridin-5-yl]phenyl]-2-oxooxazolidin-5-ylmethyl]acetamide,
5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]acetamide,
5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]acetamide,
1-[5(R)-3-[4-[2-[(1α,5α,6α)-6-amino-3-azabicyclo[3.1.0]hexan-3-yl]pyridin-5-yl]phenyl]-2-oxooxazolidin-5-ylmethyl]-1,2,3-triazole,
1-[5(R)-3-[4-[2-(1-t-butoxycarbonylaminocyclopropan-1-yl)pyridin-5-yl]-3-fluorophenyl]-2-oxooxazolidin-5-ylmethyl]-4-methyl-1,2,3-triazole,
1-[5(R)-3-[4-[2-(1-aminocyclopropan-1-yl)pyridin-5-yl]-3-fluorophenyl]-2-oxooxazolidin-5-ylmethyl]-4-methyl-1,2,3-triazole,
N-[5(S)-3-[4-[4-(1-(t-butoxycarbonyl)aminocyclopropan-1-yl)phenyl]-3-fluorophenyl]-2-oxooxazolidin-5-ylmethyl]acetamide,
N-[5(S)-3-[4-[4-(1-aminocyclopropan-1-yl)phenyl]-3-fluorophenyl]-2-oxooxazolidin-5-ylmethyl]acetamide,
N-[5(S)-3-[4-[4-(1-(t-butoxycarbonyl)aminocyclopropan-1-yl)phenyl]phenyl]-2-oxooxazolidin-5-ylmethyl]acetamide,
N-[5(S)-3-[4-[4-(1-aminocyclopropan-1-yl)phenyl]phenyl]-2-oxooxazolidin-5-ylmethyl]acetamide,
N-[5(S)-3-[4-[4-(1-(t-butoxycarbonyl)aminocyclopropan-1-yl)-3-fluorophenyl]-3-fluorophenyl]-2-oxooxazolidin-5-ylmethyl]acetamide,
N-[5(S)-3-[4-[4-(1-aminocyclopropan-1-yl)-3-fluorophenyl]-3-fluorophenyl]-2-oxooxazolidin-5-ylmethyl]acetamide,
N-[5(S)-3-[4-[4-(1-(t-butoxycarbonyl)aminocyclopropan-1-yl)-3-fluorophenyl]phenyl]-2-oxooxazolidin-5-ylmethyl]acetamide,
N-[5(S)-3-[4-[4-(1-aminocyclopropan-1-yl)-3-fluorophenyl]phenyl]-2-oxooxazolidin-5-ylmethyl]acetamide,
N-[5(S)-3-[4-[2-(1-aminocyclopropan-1-yl)-3-fluoropyridin-5-yl]-3-fluorophenyl]-2-oxooxazolidin-5-ylmethyl]acetamide,
N-[5(S)-3-[4-[4-(1-aminocyclopropan-1-yl)phenyl]-3,5-difluorophenyl]-2-oxooxazolidin-5-ylmethyl]acetamide,
N-[5(S)-3-[4-[2-(1-aminocyclopropan-1-yl)pyrimidin-5-yl]-3-fluorophenyl]-2-oxooxazolidin-5-ylmethyl]acetamide,
1-[5(R)-3-[4-[2-(1-aminocyclopropan-1-yl)pyridin-5-yl]-3,5-difluorophenyl]-2-oxooxazolidin-5-ylmethyl]-4-methyl-1,2,3-triazole,
1-[5(R)-3-[4-[2-(1-aminocyclopropan-1-yl)-3-fluoropyridin-5-yl]-3,5-difluorophenyl]-2-oxooxazolidin-5-ylmethyl]-4-methyl-1,2,3-triazole,
1-[5 (R)-3-[4-[2-(1-aminocyclopropan-1-yl)pyridin-5-yl]-3,5-difluorophenyl]-2-oxooxazolidin-5-ylmethyl]-1,2,3-triazole,
   or their enantiomer, diastereomer, or pharmaceutically acceptable salt, hydrate or prodrug thereof wherein.

Suitable subjects for the administration of the formulation of the present invention include mammals, primates, man, and other animals. In vitro antibacterial activity is predictive of in vivo activity when the compositions are administered to a mammal infected with a susceptible bacterial organism.

Using standard susceptibility tests, the compositions of the invention are determined to be active against MRSA and enterococcal infections.

The compounds of the invention are formulated in pharmaceutical compositions by combining the compounds with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, a preferred route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

The novel antibiotic compositions of this invention for human delivery per unit dosage, whether liquid or solid, comprise from about 0.01% to as high as about 99% of the cyclopropyl containing oxazolidinone compounds discussed herein, the preferred range being from about 10-60% and from about 1% to about 99.99% of one or more of other antibiotics such as those discussed herein, preferably from about 40% to about 90%. The composition will generally contain from about 125 mg to about 3.0 g of the cyclopropyl containing oxazolidinone compounds discussed herein; however, in general, it is preferable to employ dosage amounts in the range of from about 250 mg to 1000 mg and from about 200mg to about 5 g of the other antibiotics discussed herein; preferably from about 250 mg to about 1000 mg. In parenteral administration, the unit dosage will typically include the pure compound in sterile water solution or in the form of a soluble powder intended for solution, which can be adjusted to neutral pH and isotonic.

The invention described herein also includes a method of treating a bacterial infection in a mammal in need of such treatment comprising administering to said mammal the claimed composition in an amount effective to treat said infection.

Oxazolidinones have been known at times to cause side effects such as sideroblastic anemia, peripheral sensory neuropathy, optic neuropathy, seizures, thrombocytopenia, cheilosis, seborrheic dermatitis, hypo-regenerative anemia, megaloblastic anemia or normocytic anemia. The compounds of the invention may be combined with an effective amount of one or more vitamins to prevent or reduce the occurrence of oxazolidinone-associated side effects in patients. The vitamins that can be combined are vitamin B2, vitamin B6, vitaimin B12 and folic acid. The vitamins may be administered with the oxazolidinones as separate compositions or the vitamins and oxazolidinones may be present in the same composition.

Thus another aspect of this invention is a method of treating or preventing an oxazolidinone-associated side effect by administering an effective amount of the oxazolidinone of structural formula I and an effective amount of one or more of vitamin B2, vitamin B6, vitaimin B12 and folic acid to a patient in need thereof.

A further aspect of this invention relates to a method of treating or preventing oxazolidinone-associated normocyctic anemia or peripheral sensory neuropathy by administering an effective amount of vitamin B2 to a patient in need thereof.

Yet another aspect of this invention relates to a method of treating or preventing oxazolidinone-associated sideroblastic anemia, peripheral sensory neuropathy, optic neuropathy, seizures, thrombocytopenia, cheilosis, and seborrheic dermatitis by administering an effective amount of vitamin B6 to a patient in need thereof.

Still another aspect of this invention relates to a method of treating or preventing oxazolidinone-associated hypo-regenerative anemia, megaloblastic anemia by administering an effective amount of vitamin B12 and folic acid to a patient in need thereof.

Still another aspect of this invention relates to a method of treating or preventing bacterial infection by administering an effective amount of a compound of formula I and an effective amount of one or more of the group selected from the group consisting of vitamin B2, vitamin B6, vitaimin B12 and folic acid to a patient in need thereof.

The preferred methods of administration of the claimed compositions include oral and parenteral, e.g., i.v. infusion, i.v. bolus and i.m. injection formulated so that a unit dosage comprises a therapeutically effective amount of each active component or some submultiple thereof.

For adults, about 5-50 mg/kg of body weight, preferably about 250 mg to about 1000 mg per person of the cyclopropyl containing oxazolidinone antibacterial compound and about 250 mg, to about 1000 mg per person of the other antibiotic(s) given one to four times daily is preferred. More specifically, for mild infections a dose of about 250 mg two or three times daily of the cyclopropyl containing oxazolidinone antibacterial compound and about 250 mg two or three times daily of the other antibiotic is recommended. For moderate infections against highly susceptible gram positive organisms a dose of about 500 mg each of the cyclopropyl containing oxazolidinone and the other antibiotics, three or four times daily is recommended. For severe, life-threatening infections against organisms at the upper limits of sensitivity to the antibiotic, a dose of about 500-2000 mg each of the cyclopropyl-containing oxazolidinone compound and the other antibiotics, three to four times daily maybe recommended.

For children, a dose of about 5-25 mg/kg of body weight given 2, 3, or 4 times per day is preferred; a dose of 10 mg/kg is typically recommended.

The invention is further described in connection with the following non-limiting examples.

### EXAMPLE 1

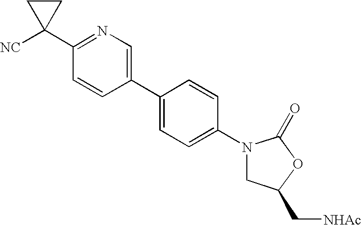

N-[5(S)-3-[4-[2-(1-Cyanocyclopropan-1-yl)pyridin-5-yl]phenyl]-2-oxooxazolidin-5-ylmethyl]acetamide.

The mixture of N-[5(S)-3-[4-(4,4,5,5-tetramethyl-1,3,2-dioxaborolyl)phenyl]-2-oxooxazolidin-5-ylmethyl]acetamide (400 mg), 5-bromo-2-(1-cyanocyclopropan-1-yl)pyridine (248 mg) and tetrakis(triphenylphosphine)palladium (0) (128 mg) in dioxane (10 mL) and 2M sodium carbonate solution (2.78 mL) was heated at 80° C. for 2 hours. Flash chromatography (silica, dichloromethane:methanol=9:1) of the mixture gave N-[5(S)-3-[4-[2-(1-cyanocyclopropan-1-yl)pyridin-5-yl]phenyl]-2-oxooxazolidin-5-ylmethyl]acetamide (298 mg).

MS (EI⁺) m/z: 376 (M⁺).

HRMS (EI⁺) for C₂₁H₂₀N₄O₃ (M⁺): calcd, 376.1535; found, 376.1533.

### EXAMPLE 2

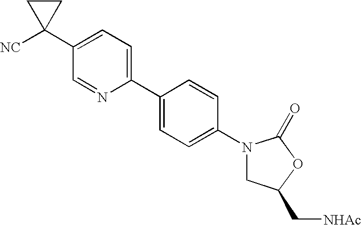

N-[5(S)-3-[4-[5-(1-Cyanocyclopropan-1-yl)pyridin-2-yl]phenyl]-2-oxooxazolidin-5-ylmethyl]acetamide.

The title compound N-[5(S)-3-[4-[5-(1-cyanocyclopropan-1-yl)pyridin-2-yl]phenyl]-2-oxooxazolidin-5-ylmethyl]acetamide (266 mg) was prepared from N-[5(S)-3-[4-(4,4,5,5-tetramethyl-1,3,2-dioxaborolyl)phenyl]-2-oxooxazolidin-5-ylmethyl]acetamide (400 mg) and 2-bromo-5-(1-cyanocyclopropan-1-yl)pyridine (248 mg) in the same manner as described for EXAMPLE 1.

MS (EI⁺) m/z: 376 (M⁺).

HRMS (EI⁺) for C₂₁H₂₀N₄O₃ (M⁺): calcd, 376.1535; found, 376.1533.

### EXAMPLE 3

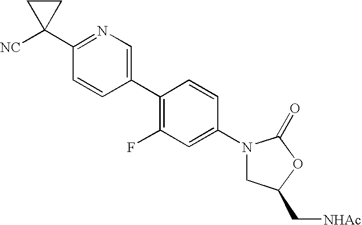

N-[5(S)-3-[4-[2-(1-Cyanocyclopropan-1-yl)pyridin-5-yl]-3-fluorophenyl]-2-oxooxazolidin-5-ylmethyl]acetamide.

The title compound N-[5(S)-3-[4-[2-(1-cyanocyclopropan-1-yl)pyridin-5-yl]-3-fluorophenyl]-2-oxooxazolidin-5-ylmethyl]acetamide (278 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (400 mg) and 5-bromo-2-(1-cyanocyclopropan-1-yl)pyridine (236 mg) in the same manner as described for EXAMPLE 1.

MS (EI⁺) m/z: 394 (M⁺)

HRMS (EI⁺) for C₂₁H₁₉FN₄O₃ (M⁺): calcd, 394.1441; found, 394.1412.

### EXAMPLE 4

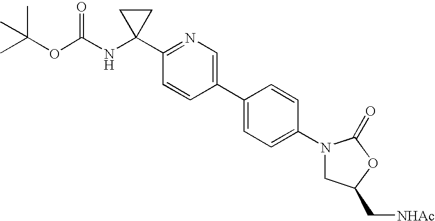

N-[5(S)-3-[4-[2-(1-(t-Butoxycarbonyl)aminocyclopropan-1-yl)pyridin-5-yl]phenyl]-2-oxooxazolidin-5-ylmethyl]acetamide.

The title compound N-[5(S)-3-[4-[2-(1-(t-butoxycarbonyl)aminocyclopropan-1-yl)pyridin-5-yl]phenyl]-2-oxooxazolidin-5-ylmethyl]acetamide (398 mg) was prepared from N-[5(S)-3-[4-(4,4,5,5-tetramethyl-1,3,2-dioxaborolyl)phenyl]-2-oxooxazolidin-5-ylmethyl]acetamide (575 mg) and 5-bromo-2-(1-(t-butoxycarbonyl)aminocyclopropan-1-yl)pyridine (500 mg) in the same manner as described for EXAMPLE 1.

MS (FAB⁺) m/z: 467 (MH⁺).

HRMS (FAB⁺) for C₂₅H₃₁N₄O₅ (MH⁺): calcd, 467.2294; found, 467.2292.

### EXAMPLE 5

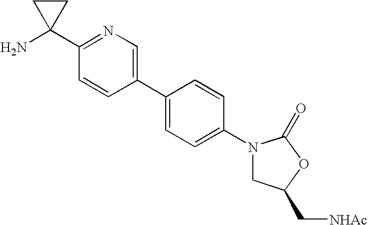

N-[5(S)-3-[4-[2-(1-Aminocyclopropan-1-yl)pyridin-5-yl]phenyl]-2-oxooxazolidin-5-ylmethyl]acetamide.

To a suspension of N-[5(S)-3-[4-[2-(1-(t-butoxycarbonyl)aminocyclopropan-1-yl)pyridin-5-yl]phenyl]-2-oxooxazolidin-5-ylmethyl]acetamide (370 mg) in dichloromethane (10 mL) was added trifluoroacetic acid (5 mL), the mixture was stirred at room temperature for 1 hour, and then concentrated in vacuo. The residue was diluted with 5% hydrochloric acid and washed with dichloromethane. The aqueous solution was adjusted to pH 10 by the addition of potassium carbonate, the resulting mixture was extracted with dichloromethane-methanol (7:1). The organic extracts were concentrated in vacuo. Flash chromatography (NH silica, dichloromethane:methanol=20:1) of the residue gave N-[5(S)-3-[4-[2-(1-aminocyclopropan-1-yl)pyridin-5-yl]phenyl]-2-oxooxazolidin-5-ylmethyl]acetamide (283 mg).

MS (EI⁺) m/z: 366 (M⁺).

HRMS (EI⁺) for C₂₀H₂₂N₄O₃ (M⁺): calcd, 366.1692; found, 366.1683.

### EXAMPLE 6

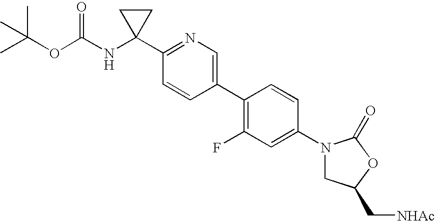

N-[5(S)-3-[4-[2-(1-(t-Butoxycarbonyl)aminocyclopropan-1-yl)pyridin-5-yl]-3-fluorophenyl]-2-oxooxazolidin-5-ylmethyl]acetamide.

The title compound N-[5(S)-3-[4-[2-(1-(t-butoxycarbonyl)aminocyclopropan-1-yl)pyridin-5-yl]-3-fluorophenyl]-2-oxooxazolidin-5-ylmethyl]acetamide (592 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (540 mg) and 5-bromo-2-(1-(t-butoxycarbonyl)aminocyclopropan-1-yl)pyridine (447 mg) in the same manner as described for EXAMPLE 1.

MS (FAB⁺) m/z: 485 (MH⁺).

HRMS (FAB⁺) for C₂₅H₃₀FN₄O₅ (MH⁺): calcd, 485.2200; found, 485.2209.

### EXAMPLE 7

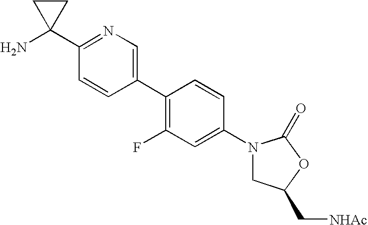

N-[5(S)-3-[4-[2-(1-Aminocyclopropan-1-yl)pyridin-5-yl]-3-fluorophenyl]-2-oxooxazolidin-5-ylmethyl]acetamide.

The title compound N-[5(S)-3-[4-[2-(1-aminocyclopropan-1-yl)pyridin-5-yl]-3-fluorophenyl]-2-oxooxazolidin-5-ylmethyl]acetamide (30.2 mg) was prepared from N-[5(S)-3-[4-[2-(1-(t-butoxycarbonyl)aminocyclopropan-1-yl)pyridin-5-yl]-3-fluorophenyl]-2-oxooxazolidin-5-ylmethyl]acetamide (50.0 mg) in the same manner as described for EXAMPLE 5.

MS (EI⁺) m/z: 384 (M⁺).

HRMS (EI⁺) for C₂₀H₂₁FN₄O₃ (M⁺): calcd, 384.1598; found, 384.1603.

### EXAMPLE 8

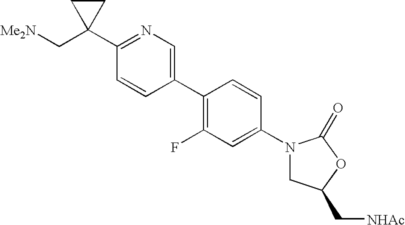

N-[5(S)-3-[4-[2-(1-(Dimethylamino)methylcyclopropan-1-yl)pyridin-5-yl]-3-fluorophenyl]-2-oxooxazolidin-5-ylmethyl]acetamide.

The title compound N-[5(S)-3-[4-[2-(1-(dimethylamino)methylcyclopropan-1-yl)pyridin-5-yl]-3-fluorophenyl]-2-oxooxazolidin-5-ylmethyl]acetamide (223 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (575 mg) and 5-bromo-2-(1-(dimethylamino)methylcyclopropan-1-yl)pyridine (337 mg) in the same manner as described for EXAMPLE 1.

MS (EI⁺) m/z: 426 (M⁺).

HRMS (EI⁺) for C₂₃H₂₇FN₄O₃ (M⁺): calcd, 426.2067; found, 426.2074.

### EXAMPLE 9

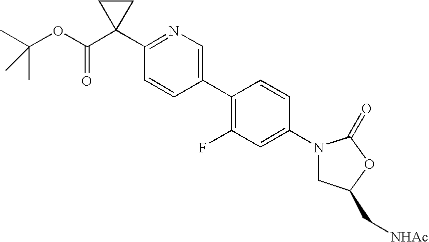

N-[5(S)-3-[4-[2-(1-t-Butoxycarbonylcyclopropan-1-yl)pyridin-5-yl]-3-fluorophenyl]-2-oxooxazolidin-5-ylmethyl]acetamide.

The title compound N-[5(S)-3-[4-[2-(1-t-butoxycarbonylcyclopropan-1-yl)pyridin-5-yl]-3-fluorophenyl]-2-oxooxazolidin-5-ylmethyl]acetamide (384 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (507 mg) and 5-bromo-2-(1-t-butoxycarbonylcyclopropan-1-yl)pyridine (400 mg) in the same manner as described for EXAMPLE 1.

MS (EI⁺) m/z: 469 (M⁺).

HRMS (EI⁺) for C₂₅H₂₈FN₃O₅ (M⁺): calcd, 469.2013; found, 469.1968.

### EXAMPLE 10

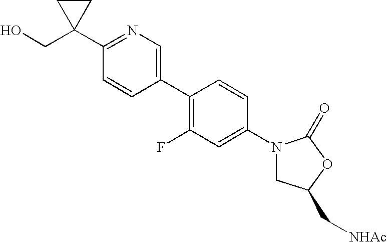

N-[5(S)-3-[4-[2-(1-Hydroxymethylcyclopropan-1-yl)pyridin-5-yl]-3-fluorophenyl]-2-oxooxazolidin-5-ylmethyl]acetamide.

The title compound N-[5(S)-3-[4-[2-(1-hydroxymethylcyclopropan-1-yl)pyridin-5-yl]-3-fluorophenyl]-2-oxooxazolidin-5-ylmethyl]acetamide (161 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (200 mg) and 5-bromo-2-(1-hydroxymethylcyclopropan-1-yl)pyridine (121 mg) in the same manner as described for EXAMPLE 1.

MS (EI⁺) m/z: 399 (M⁺).

HRMS (EI⁺) for C₂₁H₂₂FN₃O₄ (M⁺): calcd, 399.1594; found, 399.1628.

### EXAMPLE 11

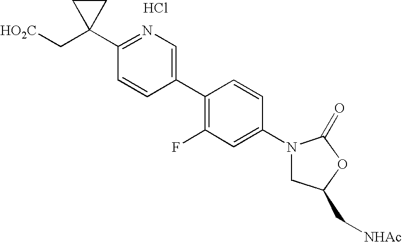

N-[5(S)-3-[4-[2-(1-Hydroxycarbonylcyclopropan-1-yl)pyridin-5-yl]-3-fluorophenyl]-2-oxooxazolidin-5-ylmethyl]acetamide Hydrochloride.

The mixture of N-[5(S)-3-[4-[2-(1-t-butoxycarbonylcyclopropan-1-yl)pyridin-5-yl]-3-fluorophenyl]-2-oxooxazolidin-5-ylmethyl]acetamide (54.8 mg) and a solution of hydrogen chloride in dioxane (4M, 1 mL) was stirred at room temperature for 4 hours, then concentrated in vacuo. Treatment with chloroform of the residue gave N-[5(S)-3-[4-[2-(1-hydroxycarbonylcyclopropan-1-yl)pyridin-5-yl]-3-fluorophenyl]-2-oxooxazolidin-5-ylmethyl]acetamide hydrochloride (49.0 mg).

MS (FAB⁺) m/z: 414 (MH⁺) (as free base).

HRMS (FAB⁺) for C₂₁H₂₁FN₃O₅ (MH⁺): calcd, 414.1465; found, 414.1512.

### EXAMPLE 12

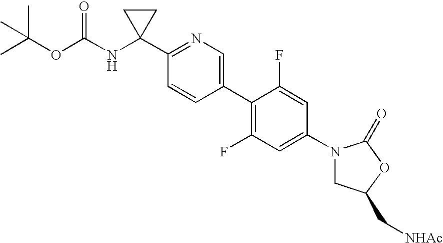

N-[5(S)-3-[4-[2-(1-(t-Butoxycarbonyl)aminocyclopropan-1-yl)pyridin-5-yl]-3,5-difluorophenyl]-2-oxooxazolidin-5-ylmethyl]acetamide.

The mixture of 5-bromo-2-(1-(t-butoxycarbonyl)aminocyclopropan-1-yl)pyridine (500 mg), bis(pinacolato)diboron (446 mg), potassium 2-ethylhexanoate (437 mg) and [1,1′-bis(diphenylphosphino)ferrocene]palladium(II) dichloride-dichloromethane adduct (130 mg) in dioxane (15 mL) was stirred at 80° C. for 1.5 hours. To this solution was added N-[5(S)-3-[3,5-difluoro-4-(trifluoromethanesulfonyl)oxyphenyl]-2-oxooxazolidin-5-ylmethyl]acetamide (601 mg), tetrakis(triphenylphosphine)palladium (0) (166 mg) and 2M sodium carbonate solution (2.2 mL), the mixture was stirred at 80° C. for 1.5 hours. Flash chromatography (NH silica, ethyl acetate:methanol=9:1) of the mixture gave N-[5(S)-3-[4-[2-(1-(t-butoxycarbonyl)aminocyclopropan-1-yl)pyridin-5-yl]-3,5-difluorophenyl]-2-oxooxazolidin-5-ylmethyl]acetamide (398 mg).

MS (FAB⁺) m/z: 503 (MH⁺).

HRMS (FAB⁺) for C₂₅H₂₉F₂N₄O₅ (MH⁺): calcd, 503.2106; found, 503.2085.

### EXAMPLE 13

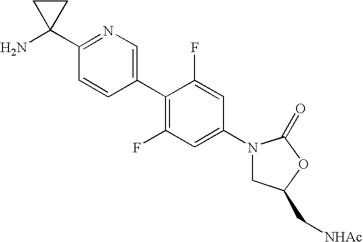

N-[5(S)-3-[4-[2-(1-Aminocyclopropan-1-yl)pyridin-5-yl]-3,5-difluorophenyl]-2-oxooxazolidin-5-ylmethyl]acetamide.

The title compound N-[5(S)-3-[4-[2-(1-aminocyclopropan-1-yl)pyridin-5-yl]-3,5-difluorophenyl]-2-oxooxazolidin-5-ylmethyl]acetamide (269 mg) was prepared from N-[5(S)-3-[4-[2-(1-(t-butoxycarbonyl)aminocyclopropan-1-yl)pyridin-5-yl]-3,5-difluorophenyl]-2-oxooxazolidin-5-ylmethyl]acetamide (398 mg) in the same manner as described for EXAMPLE 5.

MS (EI⁺) m/z: 402 (M⁺).

HRMS (EI⁺) for C₂₀H₂₀F₂N₄O₃ (M⁺): calcd, 402.1503; found, 402.1509.

### EXAMPLE 14

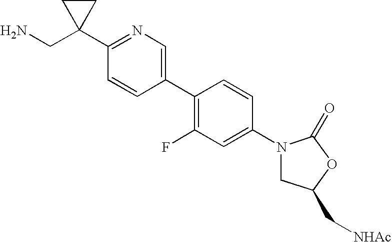

N-[5(S)-3-[4-[2-(1-Aminomethylcyclopropan-1-yl)pyridin-5-yl]-3-fluorophenyl]-2-oxooxazolidin-5-ylmethyl]acetamide.

To a suspension of N-[5(S)-3-[4-[2-(1-cyanocyclopropan-1-yl)pyridin-5-yl]-3-fluorophenyl]-2-oxooxazolidin-5-ylmethyl]acetamide (39.4 mg) in methanol (1 mL) was added cobalt dichloride hexahydrate (47.6 mg) and sodium borohydride (37.8 mg) at 0° C., the mixture was stirred at same temperature for 1 hour. The mixture was adjusted to pH 2 by addition of 1N hydrochloric acid, the resulting mixture was stirred at room temperature for 30 minutes. The mixture was adjusted to pH 10 by addition of concentrated ammonium hydroxide solution, and then concentrated in vacuo. Flash chromatography (NH silica, ethyl acetate:methanol=19:1) of the residue gave N-[5(S)-3-[4-[2-(1-aminomethylcyclopropan-1-yl)pyridin-5-yl]-3-fluorophenyl]-2-oxooxazolidin-5-ylmethyl]acetamide (25.9 mg).

MS (EI⁺) m/z: 398 (M⁺).

HRMS (EI⁺) for C₂₁H₂₃FN₄O₃ (M⁺): calcd, 398.1754; found, 398.1789.

### EXAMPLE 15

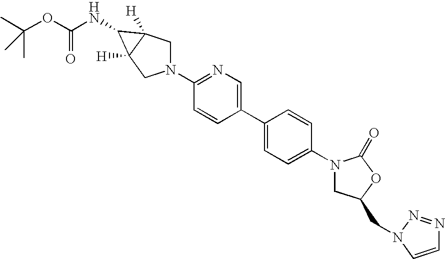

1-[5(R)-3-[4-[2-[(1α,5α,6α)-6-(N-t-Butoxycarbonyl)amino-3-azabicyclo[3.1.0]hexan-3-yl]pyridin-5-yl]phenyl]-2-oxooxazolidin-5-ylmethyl]-1,2,3-triazole.

The mixture of 1-[5(R)-3-[4-(4,4,5,5-tetramethyl-1,3,2-dioxaborolyl)phenyl]-2-oxooxazolidin-5-ylmethyl]-1,2,3-triazole (10.5 mg), 5-bromo-2-[(1α,5α,6α)-6-(N-t-butoxycarbonyl)amino-3-azabicyclo[3.1.0]hexan-3-yl]pyridine (10.0 mg), cesium carbonate (36.8 mg) and tris(dibenzylideneacetone)dipalladium(0) (6.46 mg) in dioxane (1 mL) and water (0.1 mL) was added tri(t-butyl)phosphine (2.86 mg), the mixture was stirred at 70° C. for 20 minutes. Flash chromatography (silica, ethyl acetate:methanol=6:1) of the mixture gave 1-[5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]-1,2,3-triazole (12.6 mg).

MS (FAB⁺) m/z: 518 (MH⁺).

HRMS (FAB⁺) for C₂₇H₃₂N₇O₄ (MH⁺): calcd, 518.2516; found, 518.2505.

### EXAMPLE 16

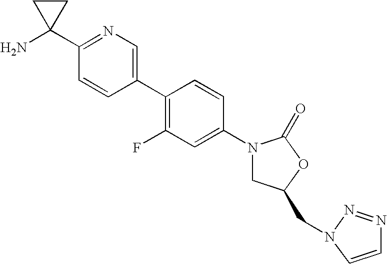

1-[5(R)-3-[4-[2-(1-Aminocyclopropan-1-yl)pyridin-5-yl]-3-fluorophenyl]-2-oxooxazolidin-5-ylmethyl]-1,2,3-triazole.

The title compound 1-[5(R)-3-[4-[2-(1-aminocyclopropan-1-yl)pyridin-5-yl]-3-fluorophenyl]-2-oxooxazolidin-5-ylmethyl]-1,2,3-triazole (261 mg) was prepared from 1-[5(R)-3-[3-fluoro-4-(4,4,5,5-tetramethyl-1,3,2-dioxaborolyl)-3-fluorophenyl]-2-oxooxazolidin-5-ylmethyl]-1,2,3-triazole (372 mg) and 5-bromo-2-(1-(t-butoxycarbonyl)aminocyclopropan-1-yl)pyridine (300 mg) in the same manner as described for EXAMPLE 1 and 5.

MS (EI⁺) m/z: 394 (M⁺).

HRMS (EI⁺) for C₂₀H₁₉FN₆O₂ (M⁺): calcd, 394.1554; found, 394.1588.

### EXAMPLE 17

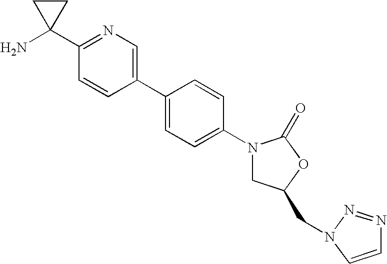

1-[5(R)-3-[4-[2-(1-Aminocyclopropan-1-yl)pyridin-5-yl]phenyl]-2-oxooxazolidin-5-ylmethyl]-1,2,3-triazole.

The title compound 1-[5(R)-3-[4-[2-(1-aminocyclopropan-1-yl)pyridin-5-yl]phenyl]-2-oxooxazolidin-5-ylmethyl]-1,2,3-triazole (231 mg) was prepared from 1-[5(R)-3-[4-(4,4,5,5-tetramethyl-1,3,2-dioxaborolyl)-3-fluorophenyl]-2-oxooxazolidin-5-ylmethyl]-1,2,3-triazole (591 mg) and 5-bromo-2-(1-(t-butoxycarbonyl)aminocyclopropan-1-yl)pyridine (500 mg) in the same manner as described for EXAMPLE 1 and 5.

MS (EI⁺) m/z: 376 (M⁺).

HRMS (EI⁺) for C₂₀H₂₀N₆O₂ (M⁺): calcd, 376.1648; found, 376.1662.

### EXAMPLE 18

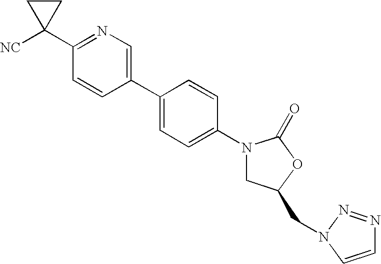

1-[5(R)-3-[4-[2-(1-Cyanocyclopropan-1-yl)pyridin-5-yl]phenyl]-2-oxooxazolidin-5-ylmethyl]-1,2,3-triazole.

The mixture of 1-[5(R)-3-(4-iodophenyl)-2-oxooxazolidin-5-ylmethyl]-1,2,3-triazole (400 mg), 2-(1-cyanocyclopropan-1-yl)pyridine-5-boric acid (264 mg) and tetrakis(triphenylphosphine)palladium (0) (125 mg) in dioxane (15 mL) and 2 M sodium carbonate solution (2.7 mL) was stirred at 80° C. for 2 hours. Flash chromatography (silica, ethyl acetate:methanol=9:1) of the mixture gave 1-[5(R)-3-[4-[2-(1-cyanocyclopropan-1-yl)pyridin-5-yl]phenyl]-2-oxooxazolidin-5-ylmethyl]-1,2,3-triazole (252 mg).

MS (EI⁺) m/z: 386 (M⁺).

HRMS (EI⁺) for C₂₁H₁₈N₆O₂ (M⁺): calcd, 386.1491; found, 386.1469.

### EXAMPLE 19

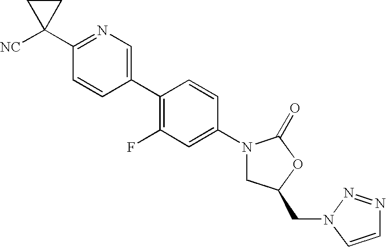

1-[5(R)-3-[4-[2-(1-Cyanocyclopropan-1-yl)pyridin-5-yl]-3-fluorophenyl]-2-oxooxazolidin-5-ylmethyl]-1,2,3-triazole.

The title compound 1-[5(R)-3-[4-[2-(1-cyanocyclopropan-1-yl)pyridin-5-yl]-3-fluorophenyl]-2-oxooxazolidin-5-ylmethyl]-1,2,3-triazole (235 mg) was prepared from 1-[5(R)-3-(3-fluoro-4-iodophenyl)-2-oxooxazolidin-5-ylmethyl]-1,2,3-triazole (400 mg) and 2-(1-cyanocyclopropan-1-yl)pyridine-5-boric acid (252 mg) in the same manner as described for EXAMPLE 18.

MS (EI⁺) m/z: 404 (M⁺).

HRMS (EI⁺) for C₂₁H₁₇FN₆O₂ (M⁺): calcd, 404.1397; found, 404.1379.

### EXAMPLE 20

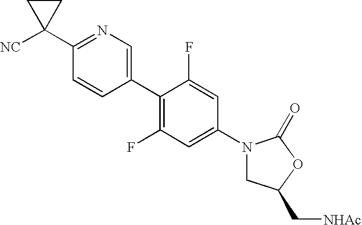

N-[5(R)-3-[4-[2-(1-Cyanocyclopropan-1-yl)pyridin-5-yl]-3,5-difluorophenyl]-2-oxooxazolidin-5-ylmethyl]acetamide.

The title compound N-[5(R)-3-[4-[2-(1-cyanocyclopropan-1-yl)pyridin-5-yl]-3,5-difluorophenyl]-2-oxooxazolidin-5-ylmethyl]acetamide (270 mg) was prepared from N-[5(S)-3-[3,5-difluoro-4-(trifluoromethanesulfonyl)oxyphenyl]-2-oxooxazolidin-5-ylmethyl]acetamide (418 mg) and 2-(1-cyanocyclopropan-1-yl)pyridine-5-boric acid (244 mg) in the same manner as described for EXAMPLE 18.

MS (EI⁺) m/z: 412 (M⁺).

HRMS (EI⁺) for C₂₁H₁₈F₂N₄O₃ (M⁺): calcd, 412.1347; found, 412.1339.

### EXAMPLE 21

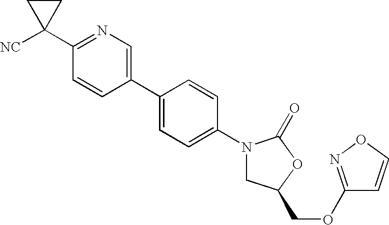

5(R)-3-[4-[2-(1-Cyanocyclopropan-1-yl)pyridin-5-yl]phenyl]-5-[(isoxazol-3-yl)oxy]methyloxazolidin-2-one.

Step 1.

5(R)-5-(t-Butyldimethylsilyloxy)methyl-3-[4-[2-(1-cyanocyclopropan-1-yl)pyridin-5-yl]phenyl]oxazolidin-2-one.

The title compound 5(R)-5-(t-butyldimethylsilyloxy)methyl-3-[4-[2-(1-cyanocyclopropan-1-yl)pyridin-5-yl]phenyl]oxazolidin-2-one (1.10 g) was prepared from 5(R)-5-(t-butyldimethylsilyloxy)methyl-3-(4-iodophenyl)oxazolidin-2-one (1.70 g) in the same manner as described for EXAMPLE 18.

MS (FAB⁺) m/z: 450 (MH⁺).

HRMS (FAB⁺) for C₂₅H₃₂N₃O₃Si (MH⁺): calcd, 450.2213; found, 450.2214;

### Step 2.

5(R)-3-[4-[2-(1-Cyanocyclopropan-1-yl)pyridin-5-yl]phenyl]-5-hydroxymethyloxazolidin-2-one.

The title compound 5(R)-3-[4-[2-(1-cyanocyclopropan-1-yl)pyridin-5-yl]phenyl]-5-hydroxymethyloxazolidin-2-one (85.1 mg) was prepared from 5(R)-5-(t-butyldimethylsilyloxy)methyl-3-[4-[2-(1-cyanocyclopropan-1-yl)pyridin-5-yl]phenyl]oxazolidin-2-one (116 mg) in the same manner as described for EXAMPLE 28.

MS (EI⁺) m/z: 335 (M⁺).

HRMS (EI⁺) for C₁₉H₁₇N₃O₃ (M⁺): calcd, 335.1270; found, 335.1286.

Step 3.

5(R)-3-[4-[2-(1-Cyanocyclopropan-1-yl)pyridin-5-yl]phenyl]-5-[(isoxazol-3-yl)oxy]methyloxazolidin-2-one.

To a suspension of 5(R)-3-[4-[2-(1-cyanocyclopropan-1-yl)pyridin-5-yl]phenyl]-5-hydroxymethyloxazolidin-2-one (50 mg), 3-hydroxyisoxazole (16.5 mg) and triphenylphosphine (58.7 mg) in tetrahydrofuran (1.5 mL) was added diisopropyl azodicarboxylate (39.2 mg), the mixture was stirred at room temperature for 30 minutes, and then concentrated in vacuo. Flash chromatography (silica, hexane:ethyl acetate=2:3) of the residue gave 5(R)-3-[4-[2-(1-cyanocyclopropan-1-yl)pyridin-5-yl]phenyl]-5-[(isoxazol-3-yl)oxy]methyloxazolidin-2-one (56.0 mg).

MS (EI⁺) m/z: 402 (M⁺).

HRMS (EI⁺) for C₂₂H₁₈N₄O₄ (M⁺): calcd, 402.1328; found, 402.1296.

### EXAMPLE 22

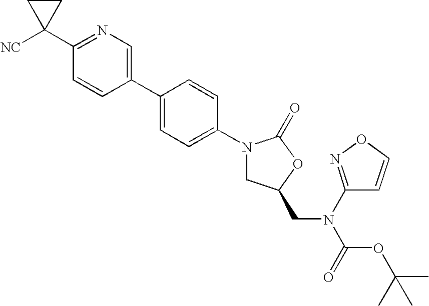

5(R)-3-[4-[2-(1-Cyanocyclopropan-1-yl)pyridinl-5-yl]phenyl]-5-[N-(t-butoxycarbonyl)-N-(isoxazol-3-yl)]aminomethyloxazolidin-2-one.

The a suspension of 5(R)-3-[4-[2-(1-cyanocyclopropan-1-yl)pyridin-5-yl]phenyl]-5-hydroxymethyloxazolidin-2-one (70.0 mg), 3-N-(t-butoxycarbonyl)aminoisoxazole (46.1 mg), and tetramethylazodicarboxamide (53.9 mg) in toluene (2 mL) was added tributylphosphine (63.3 mg), and the mixture was heated at 50° C. for 2 hours. Flash chromatography (silica, hexane:ethyl acetate=1:1) of the mixture gave 5(R)-3-[4-[2-(1-cyanocyclopropan-1-yl)pyridin-5-yl]phenyl]-5-[N-(t-butoxycarbonyl)-N-(isoxazol-3-yl)]aminomethyloxazolidin-2-one (101 mg).

MS (EI⁺) m/z: 501 (M⁺).

HRMS (EI⁺) for C₂₇H₂₇N₅O₅ (M⁺): calcd, 501.2012; found, 501.2005.

### EXAMPLE 23

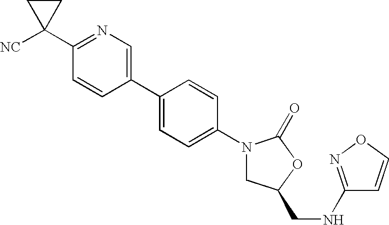

5(R)-3-[4-[2-(1-Cyanocyclopropan-1-yl)pyridin-5-yl]phenyl]-5-[N-(isoxazol-3-yl)]aminomethyloxazolidin-2-one.

The title compound 5(R)-3-[4-[2-(1-cyanocyclopropan-1-yl)pyridin-5-yl]phenyl]-5-[N-(isoxazol-3-yl)]aminomethyloxazolidin-2-one (322 mg) was prepared from 5(R)-3-[4-[2-(1-cyanocyclopropan-1-yl)pyridin-5-yl]phenyl]-5-[N-(t-butoxycarbonyl)-N-(isoxazol-3-yl)]aminomethyloxazolidin-2-one (491 mg) in the same manner as described for EXAMPLE 5.

MS (EI⁺) m/z: 401 (M⁺).

HRMS (EI⁺) for C₂₂H₁₉N₅O₃ (M⁺): calcd, 401.1488; found, 401.1515.

### EXAMPLE 24

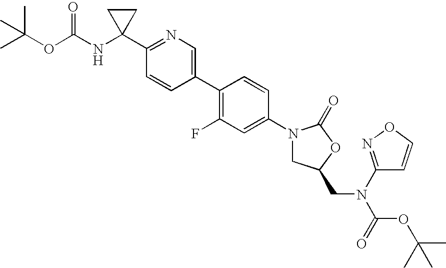

5(R)-3-[4-[2-(1-t-Butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one.

Step 1.

5(R)-3-[4-[2-(1-t-Butoxycarbonylaminocyclopropan-1-yl)pyridin-5-yl]-3-fluorophenyl]-5-(t-butyldimethylsilyloxy)methyloxazolidin-2-one.

The title compound 5(R)-3-[4-[2-(1-t-butoxycarbonylaminocyclopropan-1-yl)pyridin-5-yl]-3-fluorophenyl]-5-(t-butyldimethylsilyloxy)methyloxazolidin-2-one (76.0 mg) was prepared from 5(R)-5-(t-butyldimethylsilyloxy)methyl-3-[3-fluoro-4-(4,4,5,5-tetramethyl-1,3,2-dioxaborolyl)phenyl]oxazolidin-2-one (84.8 mg) and 5-bromo-2-(1-t-butoxycarbonylaminocyclopropan-1-yl)pyridine (58.8 mg) in the same manner as described for EXAMPLE 1.

MS (EI⁺) m/z: 557 (M⁺).

HRMS (EI⁺) for C₂₉H₄₀FN₃O₅Si (M⁺): calcd, 557.2721; found, 557.2724.

Step 2.

5(R)-3-[4-[2-(1-t-Butoxycarbonylaminocyclopropan-1-yl)pyridin-5-yl]-3-fluorophenyl]-5-hydroxymethyloxazolidin-2-one.

The title compound 5(R)-3-[4-[2-(1-t-butoxycarbonylaminocyclopropan-1-yl)pyridin-5-yl]-3-fluorophenyl]-5-hydroxymethyloxazolidin-2-one (740 mg) was prepared from 5(R)-3-[4-[2-(1-t-butoxycarbonylaminocyclopropan-1-yl)pyridin-5-yl]-3-fluorophenyl]-5-(t-butyldimethylsilyloxy)methyloxazolidin-2-one (1.32 g) in the same manner as described for EXAMPLE 28.

MS (FAB⁺) m/z: 444 (MH⁺).

HRMS (FAB⁺) for C₂₃H₂₇FN₃O₅ (MH⁺): calcd, 444.1935; found, 444.1928.

Step 3.

5(R)-3-[4-[2-(1-t-Butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one.

The title compound 5(R)-3-[4-[2-(1-t-butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one (554 mg) was prepared from 5(R)-3-[4-[2-(1-t-butoxycarbonylaminocyclopropan-1-yl)pyridin-5-yl]-3-fluorophenyl]-5-hydroxymethyloxazolidin-2-one (400 mg) and 3-N-(t-butoxycarbonyl)aminoisoxazole (203 mg) in the same manner as described for EXAMPLE 22.

MS (FAB⁺) m/z: 610 (MH⁺).

HRMS (FAB⁺) for C₃₁H₃₇FN₅O₇ (MH⁺): calcd, 610.2677; found, 610.2674.

### EXAMPLE 25

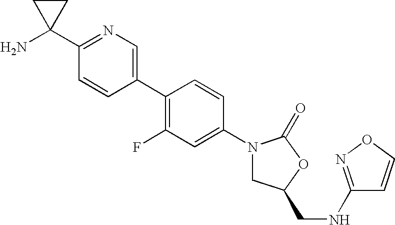

5(R)-3-[4-[2-(1-t-Aminocyclopropan-1-yl)pyridin-5-yl]-3-fluorophenyl]-5-[N-(t-butoxycarbonyl)-N-(isoxazol-3-yl)]aminomethyloxazolidin-2-one.

The title compound 5(R)-3-[4-[2-(1-t-aminocyclopropan-1-yl)pyridin-5-yl]-3-fluorophenyl]-5-[N-(t-butoxycarbonyl)-N-(isoxazol-3-yl)]aminomethyloxazolidin-2-one (224 mg) was prepared from 5(R)-3-[4-[2-(1-t-butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one (554 mg) in the same manner as described for EXAMPLE 5

MS (EI⁺) m/z: 409 (M⁺).

HRMS (EI⁺) for C₂₁H₂₀FN₅O₃(M⁺): calcd, 409.1550; found, 409.1565.

### EXAMPLE 26

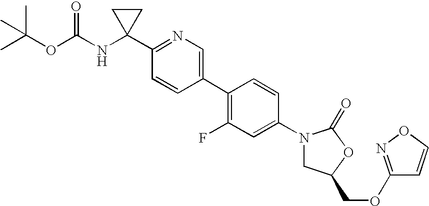

5(R)-3-[4-[2-(1-t-Butoxycarbonylaminocyclopropan-1-yl)pyridin-5-yl]phenyl]-5-[(isoxazol-3-yl)oxy]methyloxazolidin-2-one.

The title compound 5(R)-3-[4-[2-(1-t-butoxycarbonylaminocyclopropan-1-yl)pyridin-5-yl]phenyl]-5-[(isoxazol-3-yl)oxy]methyloxazolidin-2-one (335 mg) was prepared from 5(R)-3-[4-[2-(1-t-butoxycarbonylaminocyclopropan-1-yl)pyridin-5-yl]-3-fluorophenyl]-5-hydroxymethyloxazolidin-2-one (290 mg) and 3-hydroxyisoxazole (72.3 mg) in the same manner as described for EXAMPLE 21.

MS (EI⁺) m/z: 510 (M⁺).

HRMS (EI⁺) for C₂₆H₂₇FN₄O₆ (M⁺): calcd, 510.1915; found, 510.1925.

### EXAMPLE 27

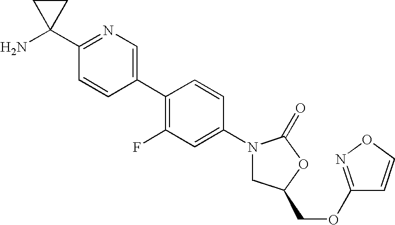

5(R)-3-[4-[2-(1-Aminocyclopropan-1-yl)pyridin-5-yl]phenyl]-5-[(isoxazol-3-yl)oxy]methyloxazolidin-2-one.

The title compound 5(R)-3-[4-[2-(1-aminocyclopropan-1-yl)pyridin-5-yl]phenyl]-5-[(isoxazol-3-yl)oxy]methyloxazolidin-2-one (115 mg) was prepared from 5(R)-3-[4-[2-(1-t-butoxycarbonylaminocyclopropan-1-yl)pyridin-5-yl]phenyl]-5-[(isoxazol-3-yl)oxy]methyloxazolidin-2-one (335 mg) in the same manner as described for EXAMPLE 5.

MS (EI⁺) m/z: 410 (M⁺).

HRMS (EI⁺) for C₂₁H₁₉FN₄O₄ (M⁺): calcd, 410.1390; found, 410.1379.

### EXAMPLE 28

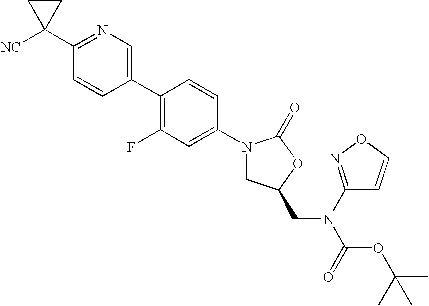

5(R)-3-[4-[2-(1-Cyanocyclopropan-1-yl)pyridin-5-yl]-3-fluorophenyl]-5-[N-(t-butoxycarbonyl)-N-(isoxazol-3-yl)]aminomethyloxazolidin-2-one.

Step 1.

5(R)-5-(t-Butyldimethylsilyloxy)methyl-3-[4-[2-(1-cyanocyclopropan-1-yl)pyridin-5-yl]-3-fluorophenyl]oxazolidin-2-one.

The title compound 5(R)-5-(t-butyldimethylsilyloxy)methyl-3-[4-[2-(1-cyanocyclopropan-1-yl)pyridin-5-yl]-3-fluorophenyl]oxazolidin-2-one (59.4 mg) was prepared from 5(R)-5-(t-butyldimethylsilyloxy)methyl-3-[3-fluoro-4-(4,4,5,5-tetramethyl-1,3,2-dioxaborolyl)phenyl]oxazolidin-2-one (60.8 mg) and 5-bromo-2-(1-cyanocyclopropan-1-yl)pyridine (30.0 mg) in the same manner as described for EXAMPLE 1.

MS (EI⁺) m/z: 467 (M⁺).

HRMS (EI⁺) for C₂₅H₃₀FN₃O₃Si (M⁺): calcd, 467.2040; found, 467.2047.

Step 2.

5(R)-3-[4-[2-(1-Cyanocyclopropan-1-yl)pyridin-5-yl]-3-fluorophenyl]-5-hydroxymethyloxazolidin-2-one.

To a solution of 5(R)-5-(t-butyldimethylsilyloxy)methyl-3-[4-[2-(1-Cyanocyclopropan-1-yl)pyridin-5-yl]-3-fluorophenyl]oxazolidin-2-one (54.6 mg) in tetrahydrofuran (2 mL) was added a solution of tetrabutylammonium fluoride (1 M solution, 0.14 mL) in tetrahydrofuran at room temperature, the mixture was stirred at he same temperature for 2.5 hours. The mixture was diluted with saturated ammonium chloride solution and extracted with ethyl acetate. The organic extracts were washed with brine, dried over anhydrous magnesium sulfate, and then concentrated in vacuo to give 5(R)-3-[4-[2-(1-cyanocyclopropan-1-yl)pyridin-5-yl]-3-fluorophenyl]-5-hydroxymethyloxazolidin-2-one (13 mg).

MS (EI⁺) m/z: 353 (M⁺).

HRMS (EI⁺) for C₁₉H₁₆FN₃O₃ (M⁺): calcd, 353.1176; found, 353.1197.

Step 3.

5(R)-3-[4-[2-(1-Cyanocyclopropan-1-yl)pyridin-5-yl]-3-fluorophenyl]-5-[N-(t-butoxycarbonyl)-N-(isoxazol-3-yl)]aminomethyloxazolidin-2-one.

The title compound 5(R)-3-[4-[2-(1-cyanocyclopropan-1-yl)pyridin-5-yl]-3-fluorophenyl]-5-[N-(t-butoxycarbonyl)-N-(isoxazol-3-yl)]aminomethyloxazolidin-2-one (67.5 mg) was prepared from 5(R)-3-[4-[2-(1-cyanocyclopropan-1-yl)pyridin-5-yl]-3-fluorophenyl]-5-hydroxymethyloxazolidin-2-one (50.0 mg) and 3-N-(t-butoxycarbonyl)aminoisoxazole (31.3 mg) in the same manner as described for EXAMPLE 22.

MS (EI⁺) m/z: 519 (M⁺).

HRMS (EI⁺) for C₂₇H₂₆FN₅O₅ (M⁺): calcd, 519.1918; found, 519.1938.

### EXAMPLE 29

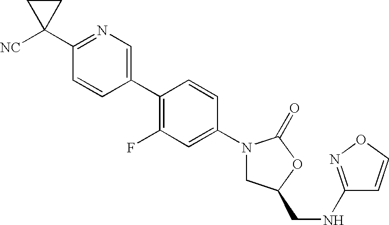

5(R)-3-[4-[2-(1-Cyanocyclopropan-1-yl)pyridin-5-yl]-3-fluorophenyl]-5-[N-(isoxazol-3-yl)]aminomethyloxazolidin-2-one.

The title compound 5(R)-3-[4-[2-(1-cyanocyclopropan-1-yl)pyridin-5-yl]-3-fluorophenyl]-5-[N-(isoxazol-3-yl)]aminomethyloxazolidin-2-one (49.8 mg) was prepared from 5(R)-3-[4-[2-(1-cyanocyclopropan-1-yl)pyridin-5-yl]-3-fluorophenyl]-5-[N-(t-butoxycarbonyl)-N-(isoxazol-3-yl)]aminomethyloxazolidin-2-one (64.2 mg) in the same manner as described for EXAMPLE 5.

MS (EI⁺) m/z: 419 (M⁺).

HRMS (EI⁺) for C₂₂H₁₈FN₅O₃ (M⁺): calcd, 419.1394; found, 419.1421.

### EXAMPLE 30

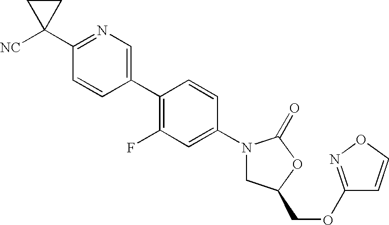

5(R)-3-[4-[2-(1-Cyanocyclopropan-1-yl)pyridin-5-yl]-3-fluorophenyl]-5-[(isoxazol-3-yl)oxy]methyloxazolidin-2-one.

The title compound 5(R)-3-[4-[2-(1-cyanocyclopropan-1-yl)pyridin-5-yl]-3-fluorophenyl]-5-[(isoxazol-3-yl)oxy]methyloxazolidin-2-one (13.6 mg) was prepared from 5(R)-3-[4-[2-(1-cyanocyclopropan-1-yl)pyridin-5-yl]-3-fluorophenyl]-5-hydroxymethyloxazolidin-2-one (13.0 mg) and 3-hydroxyisoxazole (4.1 mg) in the same manner as described for EXAMPLE 21.

MS (EI⁺) m/z: 420 (M⁺).

HRMS (EI⁺) for C₂₂H₁₇FN₄O₄ (M⁺): calcd, 420.1234; found, 420.1261.

### EXAMPLE 31

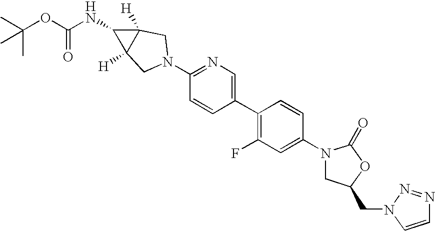

1-[5(R)-3-[4-[2-[(1α,5α,6α)-6-(N-t-Butoxycarbonyl)amino-3-azabicyclo[3.1.0]hexan-3-yl]pyridin-5-yl]-3-fluorophenyl]-2-oxooxazolidin-5-ylmethyl]-1,2,3-triazole.

The mixture of 1-[5(R)-3-[3-fluoro-4-(4,4,5,5-tetramethyl-1,3,2-dioxaborolyl)phenyl]-2-oxooxazolidin-5-ylmethyl]-1,2,3-triazole (457 mg), 5-bromo-2-[(1α,5α,6α)-6-(N-t-butoxycarbonyl)amino-3-azabicyclo[3.1.0]hexan-3-yl]pyridine (400 mg) and tetrakis(triphenylphosphine)palladium (0) (196 mg) in toluene (5.65 mL), ethanol (5.65 mL), water (2.83 mL) and 2 M sodium carbonate solution (2.82 mL) was heated at 80° C. for 3 hours. The mixture was extracted with dichloromethane. The organic extracts were washed with water and brine, dried over anhydrous magnesium sulfate, and then concentrated in vacuo. Flash chromatography (NH silica, ethyl acetate:methanol=50:1) of the residue gave 1-[5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]-1,2,3-triazole (390 mg).

MS (FAB⁺) m/z: 536 (MH⁺).

HRMS (FAB⁺) for C₂₇H₃₁FN₇O₄ (MH⁺): calcd, 536.2422; found, 536.2435.

### EXAMPLE 32

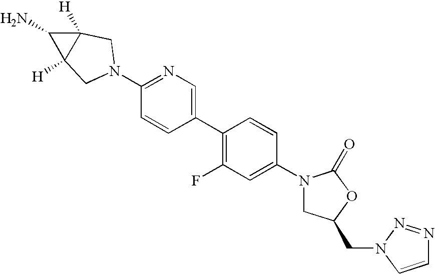

1-[5(R)-3-[4-[2-[(1α,5α,6α)-6-Amino-3-azabicyclo[3.1.0]hexan-5-yl]-3-fluorophenyl]-2-oxooxazolidin-5-ylmethyl]-1,2,3-triazole.

The title compound 1-[5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]-1,2,3-triazole (10.3 mg) was prepared from 1-[5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]-1,2,3-triazole (19.5 mg) in the same manner as described for EXAMPLE 5.

MS (FAB⁺) m/z: 436 (MH⁺).

HRMS (FAB⁺) for C₂₂H₂₃FN₇O₂ (MH⁺): calcd, 436.1897; found, 436.1919.

### EXAMPLE 33

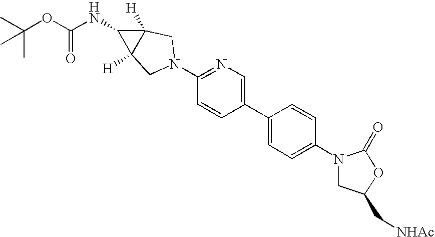

5(R)-3-[4-[2-[(1α,5α,6α)-6-(N-t-Butoxycarbonyl)amino-3-azabicyclo[3.1.0]hexan-3-yl]pyridin-5-yl]phenyl]-2-oxooxazolidin-5-ylmethyl]acetamide.

The title compound 5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]acetamide (7.00 mg) was prepared from N-[5(R)-3-[4-(4,4,5,5-tetramethyl-1,3,2-dioxaborolyl)phenyl]-2-oxooxazolidin-5-ylmethyl]acetamide (9.00 mg) and 5-bromo-2-[(1α,5α,6α)-6-(N-t-butoxycarbonyl)amino-3-azabicyclo[3.1.0]hexan-3-yl]pyridine (8.00 mg) in the same manner as described for EXAMPLE 31.

MS (EI⁺) m/z: 507 (M⁺).

HRMS (EI⁺) for C₂₇H₃₃N₅O₅ (M⁺): calcd, 507.2482; found, 507.2475.

### EXAMPLE 34

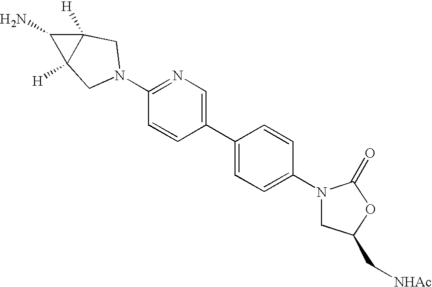

5(R)-3-[4-[2-[(1α,5α,6α)-6-Amino-3-azabicyclo[3.1.0]hexan-3-yl]pyridin-5-yl]phenyl]-2-oxooxazolidin-5-ylmethyl]acetamide.

The title compound 5(R)-3-[4-[2-[(1α,5α,6α)-6-amino-3-azabicyclo[3.1.0]hexan-3-yl]pyridin-5-yl]phenyl]-2-oxooxazolidin-5-ylmethyl]acetamide (40.0 mg) was prepared from 5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]acetamide (90.0 mg) in the same manner as described for EXAMPLE 5.

MS (EI⁺) m/z: 407 (M⁺).

HRMS (EI⁺) for C₂₂H₂₅N₅O₃ (M⁺): calcd, 407.1957; found, 407.1937.

### EXAMPLE 35

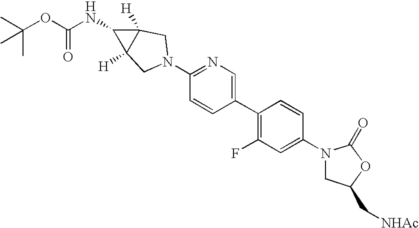

5(R)-3-[4-[2-[(1α,5α,6α)-6-(N-t-Butoxycarbonyl)amino-3-azabicyclo[3.1.0]hexan-3-yl]pyridin-5-yl]-3-fluorophenyl]-2-oxooxazolidin-5-ylmethyl]acetamide.

The title compound 5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]acetamide (50.3 mg) was prepared from N-[5(R)-3-[3-fluoro-4-(4,4,5,5-tetramethyl-1,3,2-dioxaborolyl)phenyl]-2-oxooxazolidin-5-ylmethyl]acetamide (56.7 mg), 5-bromo-2-[(1α,5α,6α)-6-(N-t-butoxycarbonyl)amino-3-azabicyclo[3.1.0]hexan-3-yl]pyridine (50.0 mg) in the same manner as described for EXAMPLE 31.

MS (EI⁺) m/z: 525 (M⁺).

HRMS (EI⁺) for C₂₇H₃₂FN₅O₅ (M⁺): calcd, 525.2387; found, 525.2408.

### EXAMPLE 36

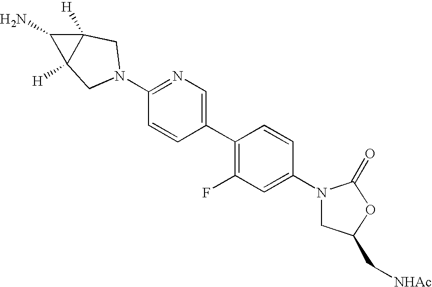

5(R)-3-[4-[2-[(1α,5α,6α)-6-Amino-3-azabicyclo[3.1.0]hexan-3-yl]pyridin-5-yl]-3-fluorophenyl]-2-oxooxazolidin-5-ylmethyl]acetamide.

The title compound 5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]acetamide (25.0 mg) was prepared from 5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]acetamide (50.0 mg) in the same manner as described for EXAMPLE 5.

MS (FAB⁺) m/z: 426 (MH⁺).

HRMS (FAB⁺) for C₂₂H₂₅FN₅O₃ (MH⁺): calcd, 426.1941; found, 426.1965.

### EXAMPLE 37

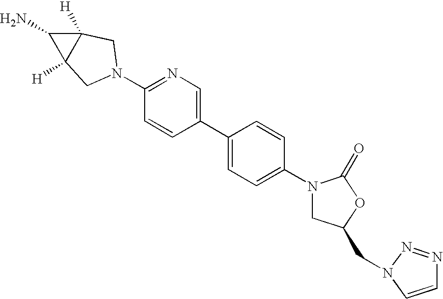

1-[5(R)-3-[4-[2-[(1α,5α,6α)-6-Amino-3-azabicyclo[3.1.0]hexan-3-yl]pyridin-5-yl]phenyl]-2-oxooxazolidin-5-ylmethyl]-1,2,3-triazole.

The title compound 1-[5(R)-3-[4-[2-[(1α,5α,6α)-6-amino-3-azabicyclo[3.1.0]hexan-3-yl]pyridin-5-yl]phenyl]-2-oxooxazolidin-5-ylmethyl]-1,2,3-triazole (10.3 mg) was prepared from 1-[5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]-1,2,3-triazole (19.5 mg) in the same manner as described for EXAMPLE 5.

MS (FAB⁺) m/z: 418 (MH⁺).

HRMS (FAB⁺) for C₂₂H₂₄N₇O₂ (MH⁺): calcd, 418.1991; found, 418.1994.

### EXAMPLE 38

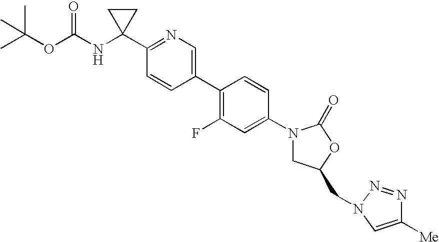

1-[5(R)-3-[4-[2-(1-t-Butoxycarbonylaminocyclopropan-1-yl)pyridin-5-yl]-3-fluorophenyl]-2-oxooxazolidin-5-ylmethyl]-4-methyl-1,2,3-triazole.

The title compound 1-[5(R)-3-[4-[2-(1-t-butoxycarbonylaminocyclopropan-1-yl)pyridin-5-yl]-3-fluorophenyl]-2-oxooxazolidin-5-ylmethyl]-4-methyl-1,2,3-triazole (465 mg) was prepared from 1-[5(R)-3-[3-fluoro-4-(4,4,5,5-tetramethyl-1,3,2-dioxaborolyl)phenyl]-2-oxooxazolidin-5-ylmethyl]-4-methyl-1,2,3-triazole (570 mg) and 5-bromo-2-(1-(t-butoxycarbonyl)aminocyclopropan-1-yl)pyridine (444 mg) in the same manner as described for EXAMPLE 1.

MS (EI⁺) m/z: 508 (M⁺).

HRMS (EI⁺) for C₂₆H₂₉FN₆O₄ (M⁺): calcd, 508.2234; found, 508.2272.

### EXAMPLE 39

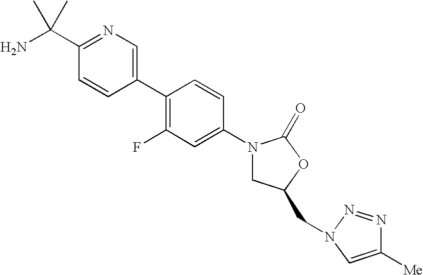

1-[5(R)-3-[4-[2-(1-Aminocyclopropan-1-yl)pyridin-5-yl]-3-fluorophenyl]-2-oxooxazolidin-5-ylmethyl]-4-methyl-1,2,3-triazole.

The title compound 1-[5(R)-3-[4-[2-(1-aminocyclopropan-1-yl)pyridin-5-yl]-3-fluorophenyl]-2-oxooxazolidin-5-ylmethyl]-4-methyl-1,2,3-triazole (210 mg) was prepared from 1-[5(R)-3-[4-[2-(1-t-butoxycarbonylaminocyclopropan-1-yl)pyridin-5-yl]-3-fluorophenyl]-2-oxooxazolidin-5-ylmethyl]-4-methyl-1,2,3-triazole (365 mg) in the same manner as described for EXAMPLE 5.

MS (EI⁺) m/z: 408 (M⁺).

HRMS (EI⁺) for C₂₁H₂₁FN₆O₂ (M⁺): calcd, 408.1710; found, 408.1690.

### EXAMPLE 40

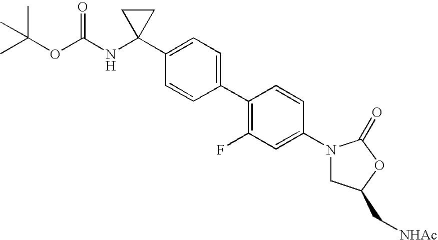

N-[5(S)-3-[4-[4-(1-(t-Butoxycarbonyl)aminocyclopropan-1-yl)phenyl]-3-fluorophenyl]-2-oxooxazolidin-5-ylmethyl]acetamide.

The title compound N-[5(S)-3-[4-[4-(1-(t-butoxycarbonyl)aminocyclopropan-1-yl)phenyl]-3-fluorophenyl]-2-oxooxazolidin-5-ylmethyl]acetamide (592 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (570 mg) and 4-bromo-1-(1-(t-butoxycarbonyl)aminocyclopropan-1-yl)benzene (471 mg) in the same manner as described for EXAMPLE 1.

MS (FAB⁺) m/z: 484 (MH⁺).

HRMS (FAB⁺) for C₂₆H₃₁FN₃O₅ (MH⁺): calcd, 484.2248; found, 484.2259.

### EXAMPLE 41

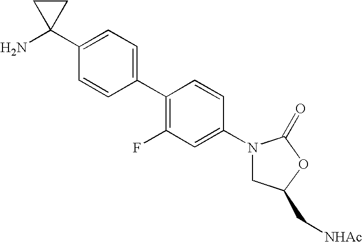

N-[5(S)-3-[4-[4-(1-Aminocyclopropan-1-yl)phenyl]-3-fluorophenyl]-2-oxooxazolidin-5-ylmethyl]acetamide.

The title compound N-[5(S)-3-[4-[4-(1-aminocyclopropan-1-yl)phenyl]-3-fluorophenyl]-2-oxooxazolidin-5-ylmethyl]acetamide (345 mg) was prepared from N-[5(S)-3-[4-[4-(1-(t-butoxycarbonyl)aminocyclopropan-1-yl)phenyl]-3-fluorophenyl]-2-oxooxazolidin-5-ylmethyl]acetamide (580 mg) in the same manner as described for EXAMPLE 5.

MS (EI⁺) m/z: 383 (M⁺).

HRMS (EI⁺) for C₂₁H₂₂FN₃O₃ (M⁺): calcd, 383.1645; found, 383.1631.

### EXAMPLE 42

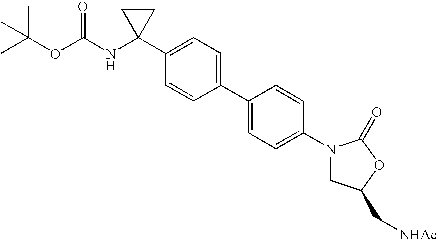

N-[5(S)-3-[4-[4-(1-(t-Butoxycarbonyl)aminocyclopropan-1-yl)phenyl]phenyl]-2-oxooxazolidin-5-ylmethyl]acetamide.

The title compound N-[5(S)-3-[4-[4-(1-(t-butoxycarbonyl)aminocyclopropan-1-yl)phenyl]phenyl]-2-oxooxazolidin-5-ylmethyl]acetamide (459 mg) was prepared from N-[5(S)-3-[4-(4,4,5,5-tetramethyl-1,3,2-dioxaborolyl)phenyl]-2-oxooxazolidin-5-ylmethyl]acetamide (450 mg) and 4-bromo-1-(1-(t-butoxycarbonyl)aminocyclopropan-1-yl)benzene (390 mg) in the same manner as described for EXAMPLE 1.

MS (FAB⁺) n/z: 466 (MH⁺).

HRMS (FAB⁺) for C₂₆H₃₂N₃O₅ (MH⁺): calcd, 466.2342; found, 466.2363.

### EXAMPLE 43

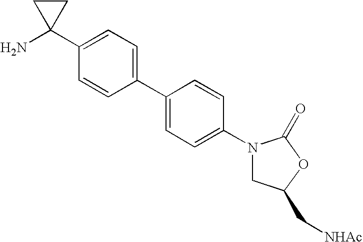

N-[5(S)-3-[4-[4-(1-Aminocyclopropan-1-yl)phenyl]phenyl]-2-oxooxazolidin-5-ylmethyl]acetamide.

The title compound N-[5(S)-3-[4-[4-(1-aminocyclopropan-1-yl)phenyl]phenyl]-2-oxooxazolidin-5-ylmethyl]acetamide (234 mg) was prepared from N-[5(S)-3-[4-[4-(1-(t-butoxycarbonyl)aminocyclopropan-1-yl)phenyl]phenyl]-2-oxooxazolidin-5-ylmethyl]acetamide (420 mg) in the same manner as described for EXAMPLE 5.

MS (FAB⁺) m/z: 366 (MH⁺).

HRMS (FAB⁺) for C₂₁H₂₄N₃O₃ (MH⁺): calcd, 366.1818; found, 366.1809.

### EXAMPLE 44

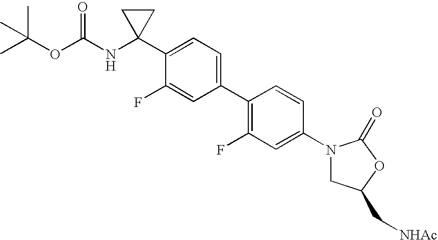

N-[5(S)-3-[4-[4-(1-(t-Butoxycarbonyl)aminocyclopropan-1-yl)-3-fluorophenyl]-3-fluorophenyl]-2-oxooxazolidin-5-ylmethyl]acetamide.

The title compound N-[5(S)-3-[4-[4-(1-(t-butoxycarbonyl)aminocyclopropan-1-yl)-3-fluorophenyl]-3-fluorophenyl]-2-oxooxazolidin-5-ylmethyl]acetamide (448 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (570 mg) and 4-bromo-1-(1-(t-butoxycarbonyl)aminocyclopropan-1-yl)-2-fluorobenzene (499 mg) in the same manner as described for EXAMPLE 1.

MS (FAB⁺) m/z: 502 (MH⁺).

HRMS (FAB⁺) for C₂₆H₃₀F₂N₃O₅ (MH⁺): calcd, 502.2154; found, 502.2113.

### EXAMPLE 45

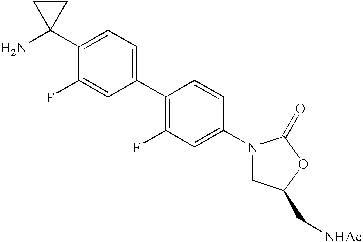

N-[5(S)-3-[4-[4-(1-Aminocyclopropan-1-yl)-3-fluorophenyl]-3-fluorophenyl]-2-oxooxazolidin-5-ylmethyl]acetamide.

The title compound N-[5(S)-3-[4-[4-(1-aminocyclopropan-1-yl)-3-fluorophenyl]-3-fluorophenyl]-2-oxooxazolidin-5-ylmethyl]acetamide (296 mg) was prepared from N-[5(S)-3-[4-[4-(1-(t-butoxycarbonyl)aminocyclopropan-1-yl)-3-fluorophenyl]-3-fluorophenyl]-2-oxooxazolidin-5-ylmethyl]acetamide (442 mg) in the same manner as described for EXAMPLE 5.

MS (FAB⁺) m/z: 402 (MH⁺).

HRMS (FAB⁺) for C₂₁H₂₂F₂N₃O₃ (MH⁺): calcd, 402.1629; found, 402.1599.

### EXAMPLE 46

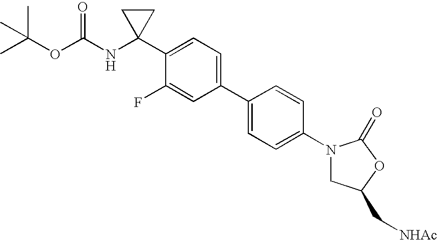

N-[5(S)-3-[4-[4-(1-(t-Butoxycarbonyl)aminocyclopropan-1-yl)-3-fluorophenyl]phenyl]-2-oxooxazolidin-5-ylmethyl]acetamide.

The title compound N-[5(S)-3-[4-[4-(1-(t-butoxycarbonyl)aminocyclopropan-1-yl)-3-fluorophenyl]phenyl]-2-oxooxazolidin-5-ylmethyl]acetamide (453 mg) was prepared from N-[5(S)-3-[4-(4,4,5,5-tetramethyl-1,3,2-dioxaborolyl)phenyl]-2-oxooxazolidin-5-ylmethyl]acetamide (500 mg) and 4-bromo-1-(1-(t-butoxycarbonyl)aminocyclopropan-1-yl)-2-fluorobenzene (458 mg) in the same manner as described for EXAMPLE 1.

MS (EI⁺) m/z: 483 (M⁺).

HRMS (EI⁺) for C₂₆H₃₀FN₃O₅ (M⁺): calcd, 483.2169; found, 483.2151.

### EXAMPLE 47

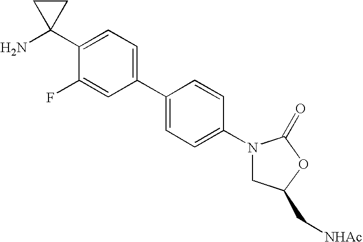

N-[5(S)-3-[4-[4-(1-Aminocyclopropan-1-yl)-3-fluorophenyl]phenyl]-2-oxooxazolidin-5-ylmethyl]acetamide.

The title compound N-[5(S)-3-[4-[4-(1-aminocyclopropan-1-yl)-3-fluorophenyl]phenyl]-2-oxooxazolidin-5-ylmethyl]acetamide (283 mg) was prepared from N-[5(S)-3-[4-[4-(1-(t-butoxycarbonyl)aminocyclopropan-1-yl)-3-fluorophenyl]phenyl]-2-oxooxazolidin-5-ylmethyl]acetamide (420 mg) in the same manner as described for EXAMPLE 5.

MS (FAB⁺) m/z: 384 (MH⁺).

HRMS (FAB⁺) for C₂₁H₂₃FN₃O₃ (MH⁺): calcd, 384.1723; found, 384.1728.

### EXAMPLE 48

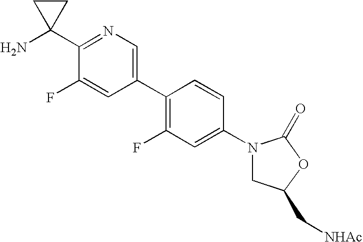

N-[5(S)-3-[4-[2-(1-Aminocyclopropan-1-yl)-3-fluoropyridin-5-yl]-3-fluorophenyl]-2-oxooxazolidin-5-ylmethyl]acetamide.

The title compound N-[5(S)-3-[4-[2-(1-aminocyclopropan-1-yl)-3-fluoropyridin-5-yl]-3-fluorophenyl]-2-oxooxazolidin-5-ylmethyl]acetamide (311 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (411 mg) and 5-bromo-2-(1-(t-butoxycarbonyl)aminocyclopropan-1-yl)-3-fluoropyridine (360 mg) in the same manner as described for EXAMPLE 1 and EXAMPLE 5.

MS (FAB⁺) m/z: 403 (MH⁺).

HRMS (FAB⁺) for C₂₀H₂₁F₂N₄O₃ (MH⁺): calcd, 403.1582; found, 403.1605.

### EXAMPLE 49

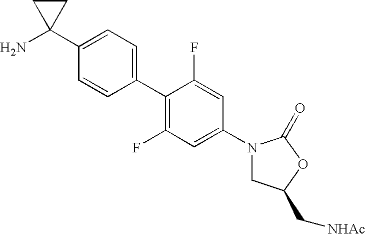

N-[5(S)-3-[4-[4-(1-Aminocyclopropan-1-yl)phenyl]-3,5-difluorophenyl]-2-oxooxazolidin-5-ylmethyl]acetamide.

The title compound N-[5(S)-3-[4-[4-(1-aminocyclopropan-1-yl)phenyl]-3,5-difluorophenyl]-2-oxooxazolidin-5-ylmethyl]acetamide (57.2 mg) was prepared from N-[5(S)-3-[3,5-difluoro-4-(trifluoromethanesulfonyl)oxyphenyl]-2-oxooxazolidin-5-ylmethyl]acetamide (121 mg) and 4-bromo-1-(1-(t-butoxycarbonyl)aminocyclopropan-1-yl)benzene (100 mg) in the same manner as described for EXAMPLE 12.

MS (FAB⁺) m/z: 402 (MH⁺).

HRMS (FAB⁺) for C₂₁H₂₂F₂N₃O₃ (MH⁺): calcd, 402.1629; found, 402.1625.

### EXAMPLE 50

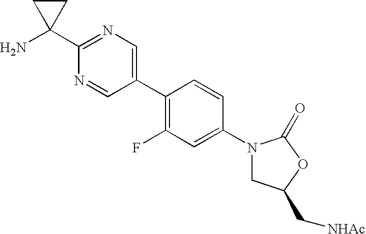

N-[5(S)-3-[4-[2-(1-Aminocyclopropan-1-yl)pyrimidin-5-yl]-3-fluorophenyl]-2-oxooxazolidin-5-ylmethyl]acetamide.

The title compound N-[5(S)-3-[4-[2-(1-aminocyclopropan-1-yl)pyrimidin-5-yl]-3-fluorophenyl]-2-oxooxazolidin-5-ylmethyl]acetamide (19.0 mg) was prepared from N-[5(S)-3-[3-fluoro-4-(4,4,5,5-tetramethyl-1,3,2-dioxaborolyl)phenyl]-2-oxooxazolidin-5-ylmethyl]acetamide (27.0 mg) and 5-bromo-2-(1-(t-butoxycarbonyl)aminocyclopropan-1-yl)pyrimidine (22.4 mg) in the same manner as described for EXAMPLE 1 and EXAMPLE 5.

MS (FAB⁺) m/z: 386 (MH⁺).

HRMS (FAB⁺) for C₁₉H₂₁FN₅O₃ (MH⁺): calcd, 386.1628; found, 386.1668.

### EXAMPLE 51

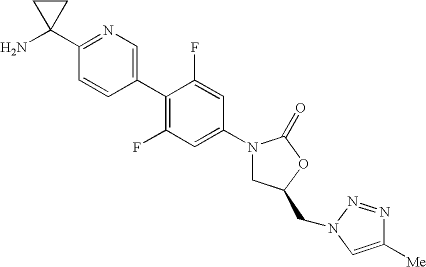

1-[5(R)-3-[4-[2-(1-Aminocyclopropan-1-yl)pyridin-5-yl]-3,5-difluorophenyl]-2-oxooxazolidin-5-ylmethyl]-4-methyl-1,2,3-triazole.

The title compound 1-[5(R)-3-[4-[2-(1-aminocyclopropan-1-yl)pyridin-5-yl]-3,5-difluorophenyl]-2-oxooxazolidin-5-ylmethyl]-4-methyl-1,2,3-triazole (253 mg) was prepared from 1-[5(R)-3-(3,5-difluoro-4-iodophenyl)-2-oxooxazolidin-5-ylmethyl]-4-methyl-1,2,3-triazole (500 mg) and 5-bromo-2-(1-(t-butoxycarbonyl)aminocyclopropan-1-yl)pyridine (447 mg) in the same manner as described for EXAMPLE 12 and EXAMPLE 5.

MS (EI⁺) m/z: 426 (M⁺).

HRMS (EI⁺) for C₂₁H₂₀F₂N₆O₂ (M⁺): calcd, 426.1616; found, 426.1646.

### EXAMPLE 52

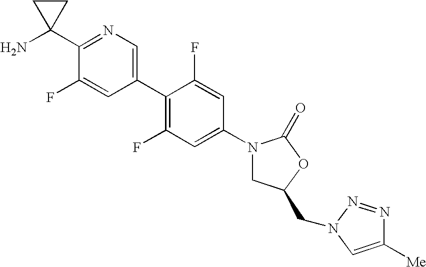

1-[5(R)-3-[4-[2-(1-Aminocyclopropan-1-yl)-3-fluoropyridin-5-yl]-3,5-difluorophenyl]-2-oxooxazolidin-5-ylmethyl]-4-methyl-1,2,3-triazole.

The title compound 1-[5(R)-3-[4-[2-(1-aminocyclopropan-1-yl)-3-fluoropyridin-5-yl]-3,5-difluorophenyl]-2-oxooxazolidin-5-ylmethyl]-4-methyl-1,2,3-triazole (298 mg) was prepared from 1-[5(R)-3-(3,5-difluoro-4-iodophenyl)-2-oxooxazolidin-5-ylmethyl]-4-methyl-1,2,3-triazole (500 mg) and 5-bromo-2-(1-(t-butoxycarbonyl)aminocyclopropan-1-yl)-3-fluoropyridine (434 mg) in the same manner as described for EXAMPLE 12 and EXAMPLE 5.

MS (EI⁺) m/z: 444 (M⁺).

HRMS (EI⁺) for C₂₁H₁₉F₃N₆O₂ (M⁺): calcd, 444.1522; found, 444.1534.

### EXAMPLE 53

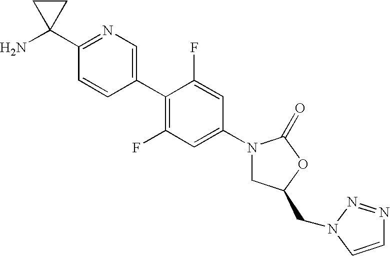

1-[5(R)-3-[4-[2-(1-Aminocyclopropan-1-yl)pyridin-5-yl]-3,5-difluorophenyl]-2-oxooxazolidin-5-ylmethyl]-1,2,3-triazole.

The title compound 1-[5(R)-3-[4-[2-(1-aminocyclopropan-1-yl)pyridin-5-yl]-3,5-difluorophenyl]-2-oxooxazolidin-5-ylmethyl]-1,2,3-triazole (252 mg) was prepared from 1-[5(R)-3-(3,5-difluoro-4-iodophenyl)-2-oxooxazolidin-5-ylmethyl]-1,2,3-triazole (460 mg) and 5-bromo-2-(1-(t-butoxycarbonyl)aminocyclopropan-1-yl)pyridine (426 mg) in the same manner as described for EXAMPLE 12 and EXAMPLE 5.

MS (EI⁺) m/z: 412 (M⁺).

HRMS (EI⁺) for C₂₀H₁₈F₂N₆O₂ (M⁺): calcd, 412.1459; found, 412.1488.

### REFERENCE EXAMPLE 1

N-[5(S)-3-[3-Fluoro-4-(4,4,5,5-tetramethyl-1,3,2-dioxaborolyl)phenyl]-2-oxooxazolidin-5-ylmethyl]acetamide.

The mixture of N-[5(S)-3-(3-fluoro-4-iodophenyl)-2-oxooxazolidin-5-ylmethyl]acetamide (2.00 g), bis(pinacolato)diboron (1.61 g), potassium acetate (1.56 g) and [1,1′-bis(diphenylphosphino)ferrocene]palladium(II) dichloride-dichloromethane adduct (432 mg) in dimethyl sulfoxide (50 mL) was heated at 80° C. for 1 hours. The mixture was diluted with water and extracted with ethyl acetate. The organic extracts were washed with brine, dried over anhydrous magnesium sulfate, and then concentrated in vacuo. Flash chromatography (silica, ethyl acetate:acetone=9:1) of the residue gave N-[5(S)-3-[3-fluoro-4-(4,4,5,5-tetramethyl-1,3,2-dioxaborolyl)phenyl]-2-oxooxazolidin-5-ylmethyl]acetamide (889 mg).

MS (EI⁺) m/z: 378 (M⁺).

HRMS (EI⁺) for C₁₈H₂₄BFN₂O₅(M⁺): calcd, 378.1762; found, 378.1779.

### REFERENCE EXAMPLE 2

N-[5(S)-3-[4-(4,4,5,5-Tetramethyl-1,3,2-dioxaborolyl)phenyl]-2-oxooxazolidin-5-ylmethyl]acetamide.

The title compound N-[5(S)-3-[4-(4,4,5,5-tetramethyl-1,3,2-dioxaborolyl)phenyl]-2-oxooxazolidin-5-ylmethyl]acetamide (92.5 mg) was prepared from N-[5(S)-3-(4-iodophenyl)-2-oxooxazolidin-5-ylmethyl]acetamide (108 mg) and bis(pinacolato)diboron (855 mg) in the same manner as described for REFERENCE EXAMPLE 1.

MS (EI⁺) m/z: 360 (M⁺).

HRMS (EI⁺) for C₁₈H₂₅BN₂O₅ (M⁺): calcd, 360.1857; found, 360.1875.

### REFERENCE EXAMPLE 3

1-[5(R)-3-[3-Fluoro-4-(4,4,5,5-tetramethyl-1,3,2-dioxaborolyl)phenyl]-2-oxooxazolidin-5-ylmethyl]-1,2,3-triazole.

The title compound 1-[5(R)-3-[3-fluoro-4-(4,4,5,5-tetramethyl-1,3,2-dioxaborolyl)phenyl]-2-oxooxazolidin-5-ylmethyl]-1,2,3-triazole (1.53 g) was prepared from 1-[5(R)-3-(3-fluoro-4-iodophenyl)-2-oxooxazolidin-5-ylmethyl]-1,2,3-triazole (2.69 g) and bis(pinacolato)diboron (1.86 g) in the same manner as described for REFERENCE EXAMPLE 1.

MS (EI⁺) m/z: 388 (M⁺).

HRMS (EI⁺) for C₁₈H₂₂BFN₄O₄ (M⁺): calcd, 388.1718; found, 388.1752.

### REFERENCE EXAMPLE 4

1-[5(R)-3-[4-(4,4,5,5-Tetramethyl-1,3,2-dioxaborolyl)phenyl]-2-oxooxazolidin-5-ylmethyl]-1,2,3-triazole.

The title compound 1-[5(R)-3-[4-(4,4,5,5-tetramethyl-1,3,2-dioxaborolyl)phenyl]-2-oxooxazolidin-5-ylmethyl]-1,2,3-triazole (147 mg) was prepared from 1-[5(R)-3-(4-iodophenyl)-2-oxooxazolidin-5-ylmethyl]-1,2,3-triazole (200 mg) and bis(pinacolato)diboron (151 mg) in the same manner as described for REFERENCE EXAMPLE 1.

MS (EI⁺) m/z: 370 (M⁺).

HRMS (EI⁺) for C₁₈H₂₃BN₄O₄ (M⁺): calcd, 370.1812; found, 370.1814.

### REFERENCE EXAMPLE 5

5(R)-5-(t-Butyldimethylsilyloxy)methyl-3-(3-fluoro-4-iodophenyl)oxazolidin-2-one.

To a solution of 5(R)-3-(3-fluoro-4-iodophenyl)-5-hydroxymethyloxazolidin-2-one (3.00 g) in dichloromethane (30 mL) was added imidazole (1.33 g) and t-butyldimethylsilyl chloride (1.48 g) at 0° C., the mixture was stirred at room temperature for 2 hours. The mixture was washed with water, 2N hydrochloric acid, saturated sodium hydrogencarbonate solution and brine, dried over anhydrous magnesium sulfate, and then concentrated in vacuo to give 5(R)-5-(t-butyldimethylsilyloxy)methyl-3-(3-fluoro-4-iodophenyl)oxazolidin-2-one (3.66 g).

MS (EI⁺) m/z: 451 (M⁺).

HRMS (EI⁺) for C₁₆H₂₃FINO₃Si(M⁺): calcd, 451.0476; found, 451.0511.

### REFERENCE EXAMPLE 6

5(R)-5-(t-Butyldimethylsilyloxy)methyl-3-[3-fluoro-4-(4,4,5,5-tetramethyl-1,3,2-dioxaborolyl)phenyl]oxazolidin-2-one.

The title compound 5(R)-5-(t-butyldimethylsilyloxy)methyl-3-[3-fluoro-4-(4,4,5,5-tetramethyl-1,3,2-dioxaborolyl)phenyl]oxazolidin-2-one (64.4 mg) was prepared from 5(R)-5-(t-butyldimethylsilyloxy)methyl-3-(3-fluoro-4-iodophenyl)oxazolidin-2-one (100 mg) and bis(pinacolato)diboron (67.0 mg) in the same manner as described for REFERENCE EXAMPLE 1.

MS (CI⁺) m/z: 452 (MH⁺).

HRMS (CI⁺) for C₂₂H₃₆BFNO₅Si (MH⁺): calcd, 452.2440; found, 452.2394.

### REFERENCE EXAMPLE 7

3,5-Difluoro-4-(methoxymethyl)oxynitrobenzene.

To a solution of 2,6-difluoro-4-nitrophenol (35.0 g) in dichloromethane (300 mL) was added diisopropylethylamine (50.2 mL) and methoxymethyl chloride (17.5 mL) at 0° C., the mixture was stirred at room temperature for 2 hours. The mixture was washed with water, 5% sodium hydrogencarbonate solution and brine, dried over anhydrous magnesium sulfate, and then concentrated in vacuo. Flash chromatography (silica, hexane:ethyl acetate=9:1) of the residue gave 3,5-difluoro-4-(methoxymethyl)oxynitrobenzene (35.2 g).

¹H NMR (CDCl₃) δ 3.59 (d, J=1.5 Hz, 3H), 5.30 (s, 2H), 7.83-7.91 (m, 2H).

### REFERENCE EXAMPLE 8

4-Benzyloxycarbonylamino-2,6-difluoro-1-(methoxymethyl)oxybenzene.

A suspension of 3,5-difluoro-4-(methoxymethyl)oxynitrobenzene (35.0 g) and palladium catalyst (10% on charcoal, 3.00 g) in methanol (250 mL) ) was hydrogenated at 1 atm for 2 hours at room temperature. After filtration of the catalyst, the filtrate was concentrated in vacuo to give 4-amino-2,6-difluoro-1-(methoxymethyl)oxybenzene. This was used in the next step without further purification. To a solution of crude 4-amino-2,6-difluoro-1-(methoxymethyl)oxybenzene thus obtained in tetrahydrofuran (500 mL) was successively added sodium hydrogencarbonate (17.4 g), water (100 mL) and benzyl chloroformate (30.0 g) at 0° C., and the mixture was stirred at room temperature for 15 minutes. The mixture was diluted with saturated sodium hydrogencarbonate solution and extracted with ethyl acetate. The organic extracts were washed with brine, dried over anhydrous magnesium sulfate, and then concentrated in vacuo. Flash chromatography (silica, hexane:ethyl acetate=6:1) of the residue gave 4-benzyloxycarbonylamino-2,6-difluoro-1-(methoxymethyl)oxybenzene (49.10 g).

MS (EI⁺) m/z: 323 (M⁺).

HRMS (EI⁺) for C₁₆H₁₅F₂NO₄ (M⁺): calcd, 323.0969; found, 323.0963.

### REFERENCE EXAMPLE 9

5(R)-3-[3,5-Difluoro-4-(methoxymethyl)oxyphenyl]-5-hydroxymethyloxazolidin-2-one.

To a solution of 4-benzyloxycarbonylamino-2,6-difluoro-1-(methoxymethyl)oxybenzene (46.3 g) in dry tetrahydrofuran (400 mL) was added a solution of n-butyllithium in hexane (1.6 M, 90.0 mL) at −78° C., and the mixture was stirred at the same temperature for 30 minutes. (R)-Glycidyl butyrate (20.3 mL) was added to the mixture at −78° C. and the mixture was allowed to stand at room temperature for 3 hours. After quenching the reaction with the addition of aqueous ammonium chloride solution, the mixture was extracted with ethyl acetate. The organic extracts were washed with brine, dried over anhydrous magnesium sulfate, filtered, and then concentrated in vacuo. To a solution of the residue in methanol (300 mL) was added potassium carbonate (20.0 g), the mixture was stirred at room temperature for 30 minutes, and then concentrated in vacuo. After dilution of the residue with water, the mixture was extracted with ethyl acetate. The organic extracts were washed with brine, dried over anhydrous magnesium sulfate, filtered, and then concentrated in vacuo. Flash chromatography (silica, hexane:ethyl acetate=1:4) of the residue gave 5(R)-3-[3,5-difluoro-4-(methoxymethyl)oxyphenyl]-5-hydroxymethyloxazolidin-2-one (36.1 g).

MS (EI⁺) m/z: 289 (M⁺).

HRMS (EI⁺) for C₁₂H₁₃F₂NO₅ (M⁺): calcd, 289.0762; found, 289.0743.

### REFERENCE EXAMPLE 10

N-[5(S)-3-[3,5-Difluoro-4-(methoxymethyl)oxyphenyl]-2-oxooxazolidin-5-ylmethyl]acetamide.

To a solution of 5(R)-3-[3,5-difluoro-4-(methoxymethyl)oxyphenyl]-5-hydroxymethyloxazolidin-2-one (5.00 g) in dichloromethane (20 mL) were successively added triethylamine (4.82 mL) and methanesulfonyl chloride (2.53 mL) at 0° C., and the mixture was stirred at the same temperature for 1 hour. The mixture was washed with water, dried over anhydrous magnesium sulfate, filtered, and then concentrated in vacuo to give 5(R)-3-[3,5-difluoro-4-(methoxymethyl)oxyphenyl]-5-methanesulfonyloxymethyloxazolidin-2-one. This was used in the next step without further purification. The mixture of crude 5(R)-3-[3,5-difluoro-4-(methoxymethyl)oxyphenyl]-5-methanesulfonyloxymethyloxazolidin-2-one thus obtained and sodium azide (3.93 g) in N,N-dimethylformamide (20 mL) was heated at 60° C. for 8 hours, and then concentrated in vacuo. The residue was diluted with ethyl acetate and washed with water and brine. The organic extracts were dried over anhydrous magnesium sulfate, filtered, and then concentrated in vacuo to give 5(R)-azidomethyl-3-[3,5-difluoro-4-(methoxymethyl)oxyphenyl]oxazolidin-2-one (5.43 g). This was used in the next step without further purification. A suspension of 5(R)-azidomethyl-3-[3,5-difluoro-4-(methoxymethyl)oxyphenyl]oxazolidin-2-one (3.53 g) and Lindlar catalyst (5% palladium on CaCO3 partially poisoned with lead, 700 mg) in methanol (110 mL) was hydrogenated at 1 atm for 6 hours at room temperature. After filtration of the catalyst, the filtrate was concentrated in vacuo. To a solution of the residue in tetrahydrofuran (15 mL) was added triethylamine (6.30 mL) and acetic anhydride (2.10 mL) at room temperature, and the mixture was stirred at the same temperature for 2 hours. After quenching the reaction by the addition of saturated sodium hydrogencarbonate solution, the mixture was extracted with ethyl acetate. The organic extracts were dried over anhydrous magnesium sulfate, filtered, and then concentrated in vacuo. Flash chromatography (silica, ethyl acetate) of the residue gave N-[5(S)-3-[3,5-difluoro-4-(methoxymethyl)oxyphenyl]-2-oxooxazolidin-5-ylmethyl]acetamide (3.45 g).

MS (EI⁺) m/z: 330 (M⁺).

HRMS (EI⁺) for C₁₄H₁₆F₂N₂O₅ (M⁺): calcd, 330.1027; found, 330.1001.

### REFERENCE EXAMPLE 11

N-[5(S)-3-(3,5-Difluoro-4-hydroxyphenyl)-2-oxooxazolidin-5-ylmethyl]acetamide.

To a solution of N-[5(S)-3-[3,5-difluoro-4-(methoxymethyl)oxyphenyl]-2-oxooxazolidin-5-ylmethyl]acetamide (200 mg) in methanol (5 mL) was added concentrated hydrochloric acid (0.50 mL), the mixture was stirred at room temperature for 1 day, and then concentrated in vacuo. Treatment with water of the residue gave N-[5(S)-3-(3,5-difluoro-4-hydroxyphenyl)-2-oxooxazolidin-5-ylmethyl]acetamide (144 mg).

MS (EI⁺) m/z: 286 (M⁺).

HRMS (EI⁺) for C₁₂H₁₂F₂N₂O₄ (M⁺): calcd, 286.0765; found, 286.0747.

### REFERENCE EXAMPLE 12

N-[5(S)-3-[3,5-Difluoro-4-(trifluoromethanesulfonyl)oxyphenyl]-2-oxooxazolidin-5-ylmethyl]acetamide.

To a solution of N-[5(S)-3-(3,5-difluoro-4-hydroxyphenyl)-2-oxooxazolidin-5-ylmethyl]acetamide (2.70 g) in pyridine (15 mL) was added triflic anhydride (2.38 mL) at 0° C., the mixture was stirred at room temperature for 12 hours. After dilution of the mixture with water, the mixture was extracted with ethyl acetate. The organic extracts were washed with 5% hydrochloric acid and brine, dried over anhydrous magnesium sulfate, filtered, and then concentrated in vacuo. Flash chromatography (silica, ethyl acetate:methanol=19: 1) of the residue gave N-[5(S)-3-[3,5-difluoro-4-(trifluoromethanesulfonyl)oxyphenyl]-2-oxooxazolidin-5-ylmethyl]acetamide (3.48 g).

MS (EI⁺) m/z: 418 (M⁺).

HRMS (EI⁺) for C₁₃H₁₁F₅N₂O₆S (M⁺): calcd, 418.0258; found, 418.0210.

### REFERENCE EXAMPLE 13

1-(5-Bromopyridin-2-yl)-1-cyclopropanecarbonitrile.

The mixture of 5-bromo-2-cyanomethylpyridine (6.00 g), triethylbenzylammonium chloride (6.94 g), 1,2-dibromoethane (3.94 mL) and 50% sodium hydroxide solution (150 mL) was stirred at 80° C. for 1 hour. After dilution of the mixture with water, the resulting precipitates were collected by filtration. Flash chromatography (silica, hexane:ethyl acetate=6:1) of the precipitates gave 1-(5-bromopyridin-2-yl)-1-cyclopropanecarbonitrile (6.21 g).

MS (CI⁺) m/z: 223 (MH⁺).

HRMS (CI⁺) for C₉H₈BrN₂ (MH⁺): calcd, 222.9871; found, 222.9853.

### REFERENCE EXAMPLE 14

1-(5-Bromopyridin-2-yl)-1-cyclopropanecarboxylic Acid.

A solution of 1-(5-bromopyridin-2-yl)-1-cyclopropanecarbonitrile (3.00 g) in ethanol (60 mL) and 25 % sodium hydroxide solution (20 mL) was heated under reflux for 10 hours, and concentrated in vacuo. After dilution of the residue with water, the mixture was adjusted to pH 3 by addition of 5% hydrochloric acid and extracted with chloroform. The organic extracts were dried over anhydrous magnesium sulfate, filtered, and then concentrated in vacuo to give 1-(5-bromopyridin-2-yl)-1-cyclopropanecarboxylic acid (3.19 g).

MS (CI⁺) m/z: 242 (MH⁺).

HRMS (CI⁺) for C₉H₉BrNO₂ (MH⁺): calcd, 241.9817; found, 241.9849.

### REFERENCE EXAMPLE 15

1-(5-Bromopyridin-2-yl)-1-t-butoxycarbonylaminocyclopropane.

To a solution of 1-(5-bromopyridin-2-yl)-1-cyclopropanecarboxylic acid (1.20 g) in dichloromethane (24 mL) was added triethylamine (1.04 mL) and diphenylphosphoryl azide (1.60 mL) at room temperature, the mixture was stirred at the same temperature for 1 hour, and then concentrated in vacuo. After dilution of the residue with toluene, the mixture was washed with water, dried over anhydrous magnesium sulfate, filtered, and then concentrated in vacuo. A solution of the residue in xylene (40 mL) was stirred at 120° C. for 2 hours. After addition t-butanol (5 mL) to the mixture, the mixture was stirred at 140° C. for 16 hours, and then concentrated in vacuo. Flash chromatography (silica, hexane:ether=2:1) of the residue gave 1-(5-bromopyridin-2-yl)-1-t-butoxycarbonylaminocyclopropane (1.40 g).

MS (FAB⁺) m/z: 313 (MH⁺).

HRMS (FAB⁺) for C₁₃H₁₈BrN₂O₂ (MH⁺): calcd, 313.0552; found, 313.0569.

### REFERENCE EXAMPLE 16

t-Butyl 1-(5-Bromopyridin-2-yl)-1-cyclopropanecarboxylate.

To a solution of 1-(5-bromopyridin-2-yl)-1-cyclopropanecarboxylic acid (2.00 g) in t-butanol (40 mL) was added a solution of di-t-butyl dicarbonate (2.71 g) in t-butanol (20 mL) and 4-(dimethylamino)pyridine (505 mg) at room temperature, the mixture was stirred at the same temperature for 6 hours, and concentrated in vacuo. After dilution of the residue with 10% potassium carbonate solution, the mixture was extracted with ethyl acetate. The organic extracts were dried over anhydrous magnesium sulfate, filtered, and then concentrated in vacuo. Flash chromatography (silica, hexane:ethyl acetate=5:1) of the residue gave t-butyl 1-(5-bromopyridin-2-yl)-1-cyclopropanecarboxylate (1.38 g).

MS (EI⁺) m/z: 297 (M⁺).

HRMS (EI⁺) for C₁₃H₁₆BrNO₂ (M⁺): calcd, 297.0364; found, 297.0329.

### REFERENCE EXAMPLE 17

5-Bromo-2-(1-hydroxymethylcyclopropan-1-yl)pyridine.

To a solution of 1-(5-bromopyridin-2-yl)-1-cyclopropanecarboxylic acid (150 mg) in tetrahydrofuran (4 mL) was added triethylamine (104 μL) and ethyl chloroformate (65.0 μL) at 0° C., the mixture was stirred at the same temperature for 30 minutes. A solution of sodium borohydride (234 mg) in water (3 mL) added to the resulting mixture at 0° C., the mixture was stirred at room temperature for 30 minutes. After quenching the reaction by addition of 1 N hydrochloric acid, the mixture was adjusted to pH 8 by addition of sodium hydrogencarbonate and extracted with ethyl acetate. The organic extracts were dried over anhydrous magnesium sulfate, filtered, and then concentrated in vacuo. Flash chromatography (silica, hexane:ethyl acetate=3:2) of the residue gave 5-bromo-2-(1-hydroxymethylcyclopropan-1-yl)pyridine (131 mg).

MS (CI⁺) m/z: 228 (MH⁺).

HRMS (CI⁺) for C₉H₁₁BrNO (MH⁺): calcd, 228.0024; found, 228.0020.

### REFERENCE EXAMPLE 18

5-Bromo-2-(1-dimethylaminomethylcyclopropan-1-yl)pyridine.

To a solution of 5-bromo-2-(1-hydroxymethylcyclopropan-1-yl)pyridine (100 mg) in dichloromethane (5 mL) was added triethylamine (91.7 μL) and methanesulfonyl chloride (40.7 μL) at 0° C., the mixture was stirred at the same temperature for 1 hour. The mixture was washed with ice water, dried over anhydrous magnesium sulfate, filtered, and then concentrated in vacuo. A solution of the residue in tetrahydrofuran (2 mL) was added to a solution of dimethylamine in tetrahydrofuran (2 M, 2.2 mL) at room temperature, the mixture was stirred at 60° C. for 12 hours, and then concentrated in vacuo. After dilution of the residue with saturated sodium hydrogencarbonate solution, the mixture was extracted with ethyl acetate. The organic extracts were dried over anhydrous magnesium sulfate, filtered, and then concentrated in vacuo. Flash chromatography (NH silica, hexane:ethyl acetate=4:1) of the residue gave 5-bromo-2-(1-dimethylaminomethylcyclopropan-1-yl)pyridine (85.9 mg).

MS (EI⁺) m/z: 254 (M⁺).

HRMS (EI⁺) for C₁₁H₁₅BrN₂ (M⁺): calcd, 254.0419; found, 254.0435.

### REFERENCE EXAMPLE 19

1-(2-Bromopyridin-5-yl)-1-cyclopropanecarbonitrile.

The title compound 1-(2-bromopyridin-5-yl)-1-cyclopropanecarbonitrile (2.19 g) was prepared from 2-bromo-5-cyanomethylpyridine (2.62 g) in the same manner as described for REFERENCE EXAMPLE 13.

MS (EI⁺) m/z: 223 (M⁺).

HRMS (EI⁺) for C₉H₇BrN₂ (M⁺): calcd, 222.9793; found, 222.9794.

### REFERENCE EXAMPLE 20

2-(1-Cyanocyclopropan-1-yl)pyridine-5-boric Acid.

To a solution of 1-(5-bromopyridin-2-yl)-1-cyclopropanecarbonitrile (200 mg) and triisopropoxyborane (169 μL) in tetrahydrofuran (5 mL) was added n-butyllithium in hexane (1.6 M, 690 μL) at −78° C., the mixture was stirred at room temperature for 1 hour. After dilution of the mixture with saturated ammonium chloride solution, the mixture was extracted with ethyl acetate. The organic extracts were washed with brine, dried over anhydrous magnesium sulfate, filtered, and then concentrated in vacuo to give 2-(1-cyanocyclopropan-1-yl)pyridine-5-boric acid (170 mg).

MS (EI⁺) m/z: 510 (M⁺) (as a trimer).

HRMS (EI⁺) for C₂₇H₂₁B₃N₆O₃ (M⁺): calcd, 510.1954; found, 510.1969.

REFERENCE EXAMPLE 21

5-Bromo-2-[(1α,5α,6α)-6-(N-t-butoxycarbonyl)amino-3-azabicyclo[3.1.0]hexan-3-yl]pyridine.

To a solution of (1α,5α,6α)-6-(N-t-butoxycarbonyl)amino-3-azabicyclo[3.1.0]hexane (40.0 mg) in N,N-dimethylformamide (0.20 mL) was added triethylamine (55.8 μL) and 5-bromo-2-fluoropyridine (25.7 μL) at room temperature, the mixture was stirred at 80-90° C. for 5 hours. After dilution of the mixture with water, the mixture was extracted with ethyl acetate. The organic extracts were washed with brine, dried over anhydrous magnesium sulfate, filtered, and then concentrated in vacuo. Flash chromatography (silica, hexane:ethyl acetate=1:1) of the residue gave 5-bromo-2-[(1α,5α,6α)-6-(N-t-butoxycarbonyl)amino-3-azabicyclo[3.1.0]hexan-3-yl]pyridine (51.0 mg).

MS (EI⁺) m/z: 353 (M⁺).

HRMS (EI⁺) for C₁₅H₂₀BrN₃O₂ (M⁺): calcd, 353.0739; found, 353.0700.

### REFERENCE EXAMPLE 22

1-[5(R)-3-(3-Fluoro-4-iodophenyl)-2-oxooxazolidin-5-ylmethyl]-1,2,3-triazole.

Step 1.

5(R)-Acetoxymethyl-3-(3-fluorophenyl)oxazolidin-2-one.

To a solution of 5(R)-3-(3-fluorophenyl)-5-hydroxymethyloxazolidin-2-one (5.28 g) in tetrahydrofuran (53 mL) was added triethylamine (3.83 mL), acetic anhydride (2.55 mL) and (4-dimethylamino)pyridine (152 mg), and the mixture was stirred at room temperature for 1 hour. After quenching the reaction by the addition of 1 N hydrochloric acid, the mixture was extracted with ethyl acetate. The organic extracts were washed with brine, dried over anhydrous sodium sulfate, filtered, and then concentrated in vacuo to give crude 5(R)-acetoxymethyl-3-(3-fluorophenyl)oxazolidin-2-one (6.33 g).

Step 2.

5(R)-Acetoxymethyl-3-(3-fluoro-4-iodophenyl)oxazolidin-2-one.

To a solution of 5(R)-acetoxymethyl-3-(3-fluorophenyl)oxazolidin-2-one (6.33 g) in acetic acid (40 mL) was added iodine monochloride (1.91 mL), the mixture was stirred at room temperature for 18 hours, and then concentrated in vacuo. The resulting residue was dissolved with ethyl acetate, the mixture was washed with aqueous sodium hydrogencarbonate solution, 20% sodium sulfite solution and brine, dried over anhydrous sodium sulfate, filtered, and then concentrated in vacuo to give crude 5(R)-acetoxymethyl-3-(3-fluoro-4-iodophenyl)oxazolidin-2-one (9.48 g).

Step 3.

5(R)-3-(3-Fluoro-4-iodophenyl)-5-hydroxymethyloxazolidin-2-one.

To a solution of crude 5(R)-acetoxymethyl-3-(3-fluoro-4-iodophenyl)oxazolidin-2-one (9.48 g) in methanol (95 mL) was added potassium carbonate (6.91 g), and the mixture was stirred at room temperature for 2.5 hours. After insoluble materials were filtered off, the filtrate was concentrated in vacuo. The residue was dissolved with ethyl acetate, the mixture was washed with brine, dried over anhydrous sodium sulfate, filtered, and then concentrated in vacuo. After treating of the residue with isopropanol, the resulting precipitates were collected by filtration to give 5(R)-3-(3-fluoro-4-iodophenyl)-5-hydroxymethyloxazolidin-2-one, and the filtrate was concentrated in vacuo. Flash chromatography (silica, ethyl acetate) of the residue gave further amount of the product (total 6.24 g).

MS (EI⁺) m/z: 337 (M⁺).

¹H NMR (CDCl₃) δ 2.15 (t, J=6.4 Hz, 1H), 3.74-4.80 (m, 5H), 7.07 (dd, J=8.8, 2.4 Hz, 1H), 7.48 (dd, J=10.3, 2.4 Hz, 1H), 7.70 (dd, J=8.8, 6.8 Hz, 1H).

Step 4.

5(R)-Azidomethyl-3-(3-fluoro-4-iodophenyl)oxazolidin-2-one.

To a solution of 5(R)-3-(3-fluoro-4-iodophenyl)-5-hydroxymethyloxazolidin-2-one (2.00 g) in dichloromethane (30 mL) was added triethylamine (1.24 mL) and methanesulfonyl chloride (551 μL) at 0° C., the mixture was stirred at the same temperature for 30 minutes. The mixture was washed with ice water, dried over anhydrous magnesium sulfate, filtered, and then concentrated in vacuo. The mixture of the residue and sodium azide (964 mg) in N,N-dimethylformamide (30 mL) was stirred at 80° C. for 2 hours and concentrated in vacuo. After dilution of the residue with water, the mixture was extracted with ethyl acetate. The organic extracts were dried over anhydrous magnesium sulfate, filtered, and then concentrated in vacuo to give 5(R)-azidomethyl-3-(3-fluoro-4-iodophenyl)oxazolidin-2-one (2.18 g).

MS (EI⁺) m/z: 361 (M⁺).

HRMS (EI⁺) for C₁₀H₈FIN₄O₂ (M⁺): calcd, 361.9676; found, 361.9698.

Step 5.

1-[5(R)-3-(3-Fluoro-4-iodophenyl)-2-oxooxazolidin-5-ylmethyl]-1,2,3-triazole.

The mixture of 5(R)-azidomethyl-3-(3-fluoro-4-iodophenyl)oxazolidin-2-one (2.18 g) and 2,5-norbornadiene (6.40 mL) in dioxane (45.6 mL) was stirred at 80° C. for 2 hours, 110° C. for 4 hours, and then concentrated in vacuo to give 1-[5(R)-3-(3-fluoro-4-iodophenyl)-2-oxooxazolidin-5-ylmethyl]-1,2,3-triazole (1.70 g).

MS (EI⁺) m/z: 388 (M⁺).

HRMS (EI⁺) for C₁₂H₁₀FIN₄O₂ (M⁺): calcd, 387.9833; found, 387.9835.

### REFERENCE EXAMPLE 23

5(R)-Azidomethyl-3-(4-iodophenyl)oxazolidin-2-one.

The title compound 5(R)-azidomethyl-3-(4-iodophenyl)oxazolidin-2-one (75.3 g) was prepared from 5(R)-3-(4-iodophenyl)-5-hydroxymethyloxazolidin-2-one (70.0 g) in the same manner as described for REFERENCE EXAMPLE 22.

MS (EI⁺) m/z: 344 (M⁺).

HRMS (EI⁺) for C₁₀H₉IN₄O₂ (M⁺): calcd, 343.9770; found, 343.9740.

### REFERENCE EXAMPLE 24

1-[5(R)-3-(4-Iodophenyl)-2-oxooxazolidin-5-ylmethyl]-1,2,3-triazole.

The title compound 1-[5(R)-3-(4-iodophenyl)-2-oxooxazolidin-5-ylmethyl]-1,2,3-triazole (62.5 mg) was prepared from 5(R)-azidomethyl-3-(4-iodophenyl)oxazolidin-2-one (100 mg) in the same manner as described for REFERENCE EXAMPLE 22.

MS (EI⁺) m/z: 370 (M⁺).

HRMS (EI⁺) for C₁₂H₁₁IN₄O₂ (M⁺): calcd, 369.9927; found, 369.9919.

### REFERENCE EXAMPLE 25

5(R)-5-(t-Butyldimethylsilyloxy)methyl-3-(4-iodophenyl)oxazolidin-2-one.

The title compound 5(R)-5-(t-butyldimethylsilyloxy)methyl-3-(4-iodophenyl)oxazolidin-2-one (2.66 g) was prepared from 5(R)-3-(4-iodophenyl)-5-hydroxymethyloxazolidin-2-one (2.00 g) in the same manner as described for REFERENCE EXAMPLE 5.

MS (EI⁺) m/z: 433 (M⁺).

HRMS (EI⁺) for C₁₆H₂₄INO₃Si (M⁺): calcd, 433.0570; found, 433.0544.

### REFERENCE EXAMPLE 26

1-[5(R)-3-[3-Fluoro-4-(4,4,5,5-tetramethyl-1,3,2-dioxaborolyl)phenyl]-2-oxooxazolidin-5-ylmethyl]-4-methyl-1,2,3-triazole. The mixture of 1-[5(R)-3-(3-fluoro-4-iodophenyl)-2-oxooxazolidin-5-ylmethyl]-4-methyl-1,2,3-triazole (590 mg), bis(pinacolato)diboron (410 mg), potassium 2-ethylhexanoate (802 mg) and [1,1′-bis(diphenylphosphino)ferrocene]palladium(II) dichloride-dichloromethane adduct (120 mg) in dioxane (15 mL) was stirred at 80° C. for 1.5 hours. Flash chromatography (silica, ethyl acetate) of the mixture gave 1-[5(R)-3-[3-fluoro-4-(4,4,5,5-tetramethyl-1,3,2-dioxaborolyl)phenyl]-2-oxooxazolidin-5-ylmethyl]-4-methyl-1,2,3-triazole (581 mg).

MS (EI⁺) m/z: 402 (M⁺).

HRMS (EI⁺) for C₁₉H₂₄BFN₄O₄ (M⁺): calcd, 402.1875; found, 402.1874.

### REFERENCE EXAMPLE 27

1-[5(R)-3-(3,5-Difluoro-4-iodophenyl)-2-oxooxazolidin-5-ylmethyl]-4-methyl-1,2,3-triazole. To a solution of 5(R)-aminomethyl-3-(3,5-difluoro-4-iodophenyl)oxazolidin-2-one (100 mg) in methanol (2 mL) was added diisopropylethylamine (262 μL) and asym-dichloroacetone tosylhydrazone (108 mg) at 0° C., the mixture was stirred at room temperature for 20 hours, and concentrated in vacuo. Flash chromatography (silica, ethyl acetate) of the residue gave 1-[5(R)-3-(3,5-difluoro-4-iodophenyl)-2-oxooxazolidin-5-ylmethyl]-4-methyl-1,2,3-triazole (110 mg).

MS (EI⁺) m/z: 420 (M⁺).

HRMS (EI⁺) for C₁₃H₁₁F₂IN₄O₂ (M⁺): calcd, 420.9895; found, 420.9904.

Antibacterial Activity

The pharmaceutically-acceptable compounds of the present invention are useful antibacterial agents having a good spectrum of activity in vitro against standard bacterial strains, which are used to screen for activity against pathogenic bacteria. Notably, the pharmaceutically-acceptable compounds of the present invention show activity against vancomycin-resistant enterococci, streptococci including penicillin-resistant S. pneumoniae, methicillin-resistant S. aureus, M catarrhalis, and C. pneumoniae. The antibacterial spectrum and potency of a particular compound may be determined in a standard test system.

The following in vitro results were obtained based on an agar dilution method except for C. pneumoniae. The activity is presented as the minimum inhibitory concentration (MIC).

S. aureus and M. catarrhalis were tested on Mueller-Hinton agar, using an approximate inoculum of 1×10⁴ cfu/spot an incubation temperature of 35° C. for 24 hours. The MIC was defined as the lowest concentration at which no visible bacterial growth was observed.

Streptococci and enterococci were tested on Mueller-Hinton agar supplemented with 5% defibrinated horse blood, using an approximate inoculum of 1×10⁴ cfu/spot an incubation temperature of 35° C. in an atmosphere of 5% CO₂ for 24 hours. The MIC was defined as the lowest concentration at which no visible bacterial growth was observed.

C. pneumoniae was tested using minimum essential medium supplemented with 10% heat-inactivated fetal bovine serum, 2 mM L-glutamine, 1 mg/ml cycloheximide and non essential amino acid. HeLa 229 cells were inoculated with 10⁴ inclusion-forming units of C. pneumoniae strain per mL. Infected cells were incubated with test compounds in complete medium at 35° C. in an atmosphere of 5% CO₂ for 72 hours. Cells monolayers were fixed in methanol, stained for chlamydial inclusions with a fluorescein-conjugated anti-Chlamydia monoclonal antibody, and were observed with fluorescence microscope. The MIC was defined as the lowest concentration at which no inclusion was observed.

| | MIC (μg/ml) | | | | |
|---|---|---|---|---|---|
| Strains | example 3 | example 5 | example 18 | example 32 | Linezolid |
| Staphylococcus aureus | | | | | |
| Smith | 0.06 | 0.25 | 0.06 | 0.125 | 1 |
| CR | 0.5 | 2 | 1 | 1 | 16 |
| MR | 0.125 | 0.5 | 0.06 | 0.125 | 1 |
| Streptococcus pneumoniae | | | | | |
| IID553 | 0.125 | 0.5 | 0.125 | 0.25 | 2 |
| PRQR | 0.125 | 0.25 | 0.125 | 0.125 | 1 |
| Streptococcus pyogenes | | | | | |
| IID692 | 0.125 | 0.25 | 0.06 | 0.125 | 1 |
| Enterococcus faecium | | | | | |
| VRQR | 0.125 | 0.5 | 0.125 | 0.5 | 2 |
| Moraxella catarrhalis | | | | | |
| ATCC25238 | 0.5 | 2 | 0.5 | 2 | 4 |

CR = chloramphenicol resistant
MR = methicillin resistant
PRQR = penicillin resistant, quinolone resistant
VRQR = vancomycin resistant, quinolone resistant
NT = not tested

The invention described herein is exemplified by the following non-limiting examples. The compound data is designated in accordance to General Guidelines for Manuscript Preparation, J. Org. Chem. Vol. 66, pg. 19A, Issue 1, 2001.

1. The present invention relates to compounds of formula I: 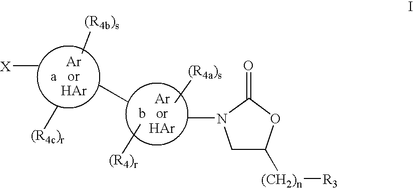
its enantiomer, diastereomer, or pharmaceutically acceptable salt, hydrate or prodrug thereof wherein:
R₁ and R₂ independently represent
i) hydrogen,
ii) (CH₂)ₙNR₅R₆,
iii) CR₇R₈R₉, C(R)₂OR₁₄, CH₂NHR₁₄,
iv) C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₃)₂, COOR₁₃, SO₂R₁₄, N(R₁₃)SO₂R₁₄, N(R₁₃)COR₁₄,
v) (C₁₋₆alkyl)CN, CN, CH═C(R)₂, (CH₂)ₚOH, C(═O)CHR₁₃, C(═NR₁₃)R₁₃, NR₁₀C(═X₁)R₁₃; or
vi) C₅₋₁₀ heterocycle optionally substituted with 1-3 groups of R₇, which may be attached through either a carbon or a heteroatom;
R₁ₐ represents (CH₂)ₙNR₅R₆, CR₇R₈R₉, C(R)₂OR₁₄, CH₂NHR₁₄, C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃,SO₂R₁₄, N(R₁₃)SO₂R₁₄, N(R₁₃)COR₁₄, (C₁₋₆alkyl)CN, CN, CH═C(R)₂, (CH₂)ₚOH, C(═O) CHR₁₃, C(═NR₁₃)R₁₃, NR₁₀C(═X₁)R₁₃; or C₅₋₁₀ heterocycle optionally substituted with 1-3 groups of R₇, which may be attached through either a carbon or a heteroatom;
X is selected from the group consisting of, 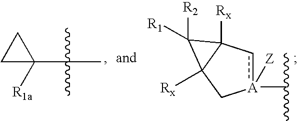
Z represents (O)ₙ, H, OH, or halogen;
A represents C (when --- is present provided Z=(O)ₙ and n=0), C (when --- is not present provided Z is H, OH or halogen), or N (when --- is not present and Z=(O)ₙ and n=1);
--- represents a bond; 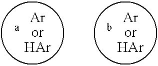
represents aryl or heteroaryl, provided that in the case of a heteroaryl, a cyclopropyl is not attached to a nitrogen atom on the ring;
Rₓ represents hydrogen or C₁₋₆ alkyl;
R₃ represent
i) NR₁₃(C═X₂)R₁₂,
ii) NR₁₃(C═X₁)R₁₂,
iii) NR₁₃SO₂R₁₄,
iv) N(R₁₃)heteroaryl,
v) NR₁₃(CHR₁₃)₀₋₄aryl,
vi) NR₁₃(CHR₁₃)₀₋₄heteroaryl,
vii) S(CHR₁₃)₀₋₄aryl,
viii) S(CHR₁₃)₀₋₄heteroaryl,
ix) O(CHR₁₃)₀₋₄aryl,
x) O(CHR₁₃)₀₋₄heteroaryl,
xi) NOH(C═X₁)R₁₂,
xii) —OC═N(OCOaryl)C₁₋₆ alkyl
xiii) —OC═N(OH)C₁₋₆ alkyl
xiv) C₅₋₁₀ heteroaryl which may be attached through either a carbon or a heteroatom; said aryl and heteroaryl optionally substituted with 1-3 groups of R₇,
R₄, R₄ₐ, R_{4b}, and R_{4c} independently represent
i) hydrogen,
ii) halogen,
iii) C₁₋₆ alkoxy, or
iv) C₁₋₆ alkyl
r and s independently are 1-3, with the provision that when (R₄ₐ)ₛ and (R₄)ᵣ or (R_{4b}) and (R_{4c})ₛ are attached to an Ar or HAr ring the sum of r and s is less than or equal to 4;
R₅ and R₆ independently represent
i) hydrogen,
ii) C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;
iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
iv) C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl;
v) arylsulfonyl optionally substituted with 1-3 of halogen, C₁₋₆ alkoxy, OH or C₁₋₆ alkyl;
vi) C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl;
vii) aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl
viii) five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
ix) C₃₋₆ cycloalkylcarbonyl optionally substituted with 1-3 groups of halogen, OH, C₁₋₆ alkoxy or CN;
x) benzoyl optionally substituted with 1-3 groups of halogen, OH, C₁₋₆ alkoxy, C₁₋₆ alkyl, CF₃, C₁₋₆ alkanoyl, amino or C₁₋₆ acylamino;
xi) pyrrolylcarbonyl optionally substituted with 1-3 of C₁₋₆ alkyl;
xii) C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or
R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
i) hydrogen, halogen, CN, CO₂R, CON(R)₂, CHO, (CH₂)₀₋₃NHAc, C(═NOR), OH, C₁₋₆ alkoxy, C₁₋₆ alkyl, alkenyl, hydroxy C₁₋₆ alkyl, (CH₂)₁₋₃NHC(O)C₁₋₆ alkyl, (CH₂)₀₋₃N(C₁₋₆ alkyl)₂
ii) (CH₂)ₙamino, (CH₂)ₙC₁₋₆ alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
i) H, CN,
ii) C₁₋₆ alkyl optionally substituted with 1-3 halogen, CN, OH, C₁₋₆ alkoxy, C₁₋₆ acyloxy, or amino,
iii) phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
X₂ represents O, S, NH or NSO₂R₁₄;
R₁₀ represents hydrogen, C₁₋₆ alkyl or CO₂R₁₅;
R₁₂ represents hydrogen, C₁₋₆ alkyl, NH₂, OR, CHF₂, CHCl₂, CR₂Cl, (CH₂)ₙSR, (CH₂)ₙCN, (CH₂)ₙSO₂R, (CH₂)ₙS(O)R, C₁₋₆ alkylamino, C₅₋₁₀ heteroaryl or C₁₋₆ dialkylamino, where said alkyl may be substituted with 1-3 groups of halo, CN, OH or C₁₋₆ alkoxy, said heteroaryl optionally substituted with 1-3 groups of R₇;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, —OCOaryl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen or C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
p represents 0-2 and
n represents 0-1.

2. A compound according to claim 1 wherein R₁ and R₂ independently represent H, NR₅R₆, CN, OH, C(R)₂OR₁₄, NHC(═X₁)N(R₁₃)₂, C(═NOH)N(R₁₃)₂, NR₁₀C(═X₁)R₁₃ or CR₇R₈R₉ and R₁ₐ represents NR₅R₆, CN, OH, C(R)₂OR₁₄, NHC(═X₁)N(R₁₃)₂, C(═NOH)N(R₁₃)₂, NR₁₀C(═X₁)R₁₃ or CR₇R₈R₉.

3. A compound according to claim 2 wherein 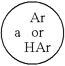
and 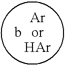
independently are phenyl, pyridine, pyrimidine, or piperidine.

4. A compound according to claim 3 wherein when X is

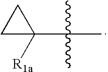

5. A compound according to claim 3 wherein X is

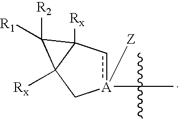

6. A compound according to claim 5 wherein A is C, --- is present and Z=(O)ₙ where n=0, A is C, --- is not present and Z=H, OH or halogen, or A is N, --- is not present and Z=(O)n where n=1.

7. A compound according to claim 6 wherein one of R₁ and R₂ is H and the other is NR₅R₆, or H and the other is NR₁₀C(═X₁)R₁₃

8. A compound according to claim 4 wherein one of R₁ₐ is CN, NR₁₀C(═X₁)R₁₃, or NR₅R₆.

9. A compound according to claim 1 wherein R₃ is NR(C═X₁)R₁₂, C₅₋₁₀ heteroaryl, NH(CH₂)₀₋₄aryl, NH(CH₂)₀₋₄heteroaryl, said aryl and heteroaryl optionally substituted with 1-3 groups of Ra.

10. A compound according to claim 9 wherein R₃ is a C₅₋₁₀ heteroaryl represented by \textcircled{N}which represents an optionally substituted aromatic heterocyclic group containing 1 to 4 nitrogen atoms and at least one double bond, and which is connected through a bond on any nitrogen.

11. A compound according to claim 1 wherein the structural formula is II: 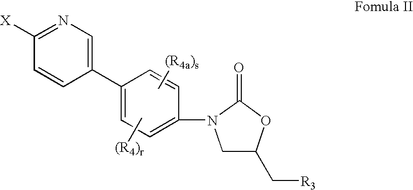
Wherein:
X is selected from the group consisting of, 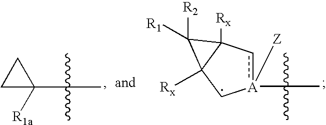
Z represents (O)ₙ, H, OH, or halogen;
A represents C (when --- is present provided Z=(O)ₙ and n=0), C (when --- is not present provided Z is H, OH or halogen), or N (when --- is not present and Z=(O)ₙ and n=1); and R₁ₐ, R₁, R₂, Rx, R₄, R₄ₐ, and R₃ are as previously described herein.

12. A compound according to claim 11 wherein R₁ₐ is CN or NR₅R₆.

13. A compound which is:
N-[5(S)-3-[4-[2-(1-cyanocyclopropan-1-yl)pyridin-5-yl]phenyl]-2-oxooxazolidin-5-ylmethyl]acetamide,
N-[5(S)-3-[4-[5-(1-cyanocyclopropan-1-yl)pyridin-2-yl]phenyl]-2-oxooxazolidin-5-ylmethyl]acetamide,
N-[5(S)-3-[4-[2-(1-cyanocyclopropan-1-yl)pyridin-5-yl]-3-fluorophenyl]-2-oxooxazolidin-5-ylmethyl]acetamide,
N-[5(S)-3-[4-[2-(1-(t-butoxycarbonyl)aminocyclopropan-1-yl)pyridin-5-yl]phenyl]-2-oxooxazolidin-5-ylmethyl]acetamide,
N-[5(S)-3-[4-[2-(1-aminocyclopropan-1-yl)pyridin-5-yl]phenyl]-2-oxooxazolidin-5-ylmethyl]acetamide,
N-[5(S)-3-[4-[2-(1-(t-butoxycarbonyl)aminocyclopropan-1-yl)pyridin-5-yl]-3-fluorophenyl]-2-oxooxazolidin-5-ylmethyl]acetamide,
N-[5(S)-3-[4-[2-(1-aminocyclopropan-1-yl)pyridin-5-yl]-3-fluorophenyl]-2-oxooxazolidin-5-ylmethyl]acetamide,
N-[5(S)-3-[4-[2-(1-(dimethylamino)methylcyclopropan-1-yl)pyridin-5-yl]-3-fluorophenyl]-2-oxooxazolidin-5-ylmethyl]acetamide,
N-[5(S)-3-[4-[2-(1-(dimethylamino)methylcyclopropan-1-yl)pyridin-5-yl]-3-fluorophenyl]-2-oxooxazolidin-5-ylmethyl]acetamide,
N-[5(S)-3-[4-[2-(1-t-butoxycarbonylcyclopropan-1-yl)pyridin-5-yl]-3-fluorophenyl]-2-oxooxazolidin-5-ylmethyl]acetamide,
N-[5(S)-3-[4-[2-(1-hydroxymethylcyclopropan-1-yl)pyridin-5-yl]-3-fluorophenyl]-2-oxooxazolidin-5-ylmethyl]acetamide,
N-[5(S)-3-[4-[2-(1-hydroxycarbonylcyclopropan-1-yl)pyridin-5-yl]-3-fluorophenyl]-2-oxooxazolidin-5-ylmethyl]acetamide hydrochloride,
N-[5(S)-3-[4-[2-(1-(t-butoxycarbonyl)aminocyclopropan-1-yl)pyridin-5-yl]-3,5-difluorophenyl]-2-oxooxazolidin-5-ylmethyl]acetamide,
N-[5(S)-3-[4-[2-(1-aminocyclopropan-1-yl)pyridin-5-yl]-3,5-difluorophenyl]-2-oxooxazolidin-5-ylmethyl]acetamide,
N-[5(S)-3-[4-[2-(1-aminomethylcyclopropan-1-yl)pyridin-5-yl]-3-fluorophenyl]-2-oxooxazolidin-5-ylmethyl]acetamide,
1-[5(R)-3-[4-[2-[(1α,5α,6α)-6-(N-t-butoxycarbonyl)amino-3-azabicyclo[3.1.0]hexan-3-yl]pyridyl-5-yl]phenyl]-2-oxooxazolidin-5-ylmethyl]-1,2,3-triazole,
1-[5(R)-3-[4-[2-(1-aminocyclopropan-1-yl)pyridin-5-yl]-3-fluorophenyl]-2-oxooxazolidin-5-ylmethyl]-1,2,3-triazole,
1-[5(R)-3-[4-[2-(1-aminocyclopropan-1-yl)pyridin-5-yl]phenyl]-2-oxooxazolidin-5-ylmethyl]-1,2,3-triazole,
1-[5(R)-3-[4-[2-(1-cyanocyclopropan-1-yl)pyridin-5-yl]phenyl]-2-oxooxazolidin-5-ylmethyl]-1,2,3-triazole,
1-[5(R)-3-[4-[2-(1-cyanocyclopropan-1-yl)pyridin-5-yl]-3-fluorophenyl]-2-oxooxazolidin-5-ylmethyl]-1,2,3-triazole,
N-[5(R)-3-[4-[2-(1-cyanocyclopropan-1-yl)pyridin-5-yl]-3,5-difluorophenyl]-2-oxooxazolidin-5-ylmethyl]acetamide,
5(R)-3-[4-[2-(1-cyanocyclopropan-1-yl)pyridin-5-yl]phenyl]-5-[(isoxazol-3-yl)oxy]methyloxazolidin-2-one,
5(R)-3-[4-[2-(1-cyanocyclopropan-1-yl)pyridin-5-yl]phenyl]-5-[N-(t-butoxycarbonyl)-N-(isoxazol-3-yl)]aminomethyloxazolidin-2-one,
5(R)-3-[4-[2-(1-cyanocyclopropan-1-yl)pyridin-5-yl]phenyl]-5-[N-(isoxazol-3-yl)]aminomethyloxazolidin-2-one,
5(R)-3-[4-[2-(1-t-butoxycarbonylaminocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-aminocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-t-butoxycarbonylaminocyclopropan-1-yl)pyridin-5-yl]phenyl]-5-[(isoxazol-3-yl)oxy]methyloxazolidin-2-one,
5(R)-3-[4-[2-(1-aminocyclopropan-1-yl)pyridin-5-yl]phenyl]-5-[(isoxazol-3-yl)oxy]methyloxazolidin-2-one,
5(R)-3-[4-[2-(1-cyanocyclopropan-1-yl)pyridin-5-yl]-3-fluorophenyl]-5-[N-(t-butoxycarbonyl)-N-(isoxazol-3-yl)]aminomethyloxazolidin-2-one,
5(R)-3-[4-[2-(1-cyanocyclopropan-1-yl)pyridin-5-yl]-3-fluorophenyl]-5-[N-(isooxazol-3-yl)]aminomethyloxazolidin-2-one,
5(R)-3-[4-[2-(1-cyanocyclopropan-1-yl)pyridin-5-yl]-3-fluorophenyl]-5-[(isoxazol-3-yl)oxy]methyloxazolidin-2-one,
1-[5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]-1,2,3-triazole, 1-[5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]-1,2,3-triazole,
5(R)-3-[4-[2-[(1α,5α,6α)-6-(N-t-butoxycarbonyl)amino-3-azabicyclo[3.1.0]hexan-3-yl]pyridin-5-yl]phenyl]-2-oxooxazolidin-5-ylmethyl]acetamide,
5(R)-3-[4-[2-[(1α,5α,6α)-6-amino-3-azabicyclo[3.1.0]hexan-3-yl]pyridin-5-yl]phenyl]-2-oxooxazolidin-5-ylmethyl]acetamide,
5(R)-3-[4-[2-[(1α,5α,6α)-6-(N-t-butoxycarbonyl)amino-3-azabicyclo[3.1.0]hexan-3-yl]pyridin-5-yl]-3-fluorophenyl]-2-oxooxazolidin-5-ylmethyl]acetamide,
5(R)-3-[4-[2-[(1α,5α,6α)-6-amino-3-azabicyclo[3.1.0]hexan-3-yl]pyridin-5-yl]-3-fluorophenyl]-2-oxooxazolidin-5-ylmethyl]acetamide,
1-[5(R)-3-[4-[2-[(1α,5α,6α)-6-amino-3-azabicyclo [3.1.0]hexan-3-yl]pyridin-5-yl]phenyl]-2-oxooxazolidin-5-ylmethyl]-1,2,3-triazole,
1-[5(R)-3-[4-[2-(1-t-butoxycarbonylaminocyclopropan-1-yl)pyridin-5-yl]-3-fluorophenyl]-2-oxooxazolidin-5-ylmethyl]-4-methyl-1,2,3-triazole,
1-[5(R)-3-[4-[2-(1-aminocyclopropan-1-yl)pyridin-5-yl]-3-fluorophenyl]-2-oxooxazolidin-5-ylmethyl]-4-methyl-1,2,3-triazole,
N-[5 (S)-3-[4-[4-(1-(t-butoxycarbonyl)aminocyclopropan-1-yl)phenyl]-3-fluorophenyl]-2-oxooxazolidin-5-ylmethyl]acetamide,
N-[5(S)-3-[4-[4-(1-aminocyclopropan-1-yl)phenyl]-3-fluorophenyl]-2-oxooxazolidin-ylmethyl]acetamide,
N-[5(S)-3-[4-[4-(1-(t-butoxycarbonyl)aminocyclopropan-1-yl)phenyl]phenyl]-2-oxooxazolidin-5-ylmethyl]acetamide,
N-[5(S)-3-[4-[4-(1-aminocyclopropan-1-yl)phenyl]phenyl]-2-oxooxazolidin-5-ylmethyl]acetamide,
N-[5(S)-3-[4-[4-(1-(t-butoxycarbonyl)aminocyclopropan-1-yl)-3-fluorophenyl]-3-fluorophenyl]-2-oxooxazolidin-5-ylmethyl]acetamide,
N-[5(S)-3-[4-[4-(1-aminocyclopropan-1-yl)-3-fluorophenyl]-3-fluorophenyl]-2-oxooxazolidin-5-ylmethyl]acetamide,
N-[5(S)-3-[4-[4-(1-(t-butoxycarbonyl)aminocyclopropan-1-yl)-3-fluorophenyl]phenyl]-2-oxooxazolidin-5-ylmethyl]acetamide,
N-[5(S)-3-[4-[4-(1-aminocyclopropan-1-yl)-3-fluorophenyl]phenyl]-2-oxooxazolidin-5-ylmethyl]acetamide,
N-[5(S)-3-[4-[2-(1-aminocyclopropan-1-yl)-3-fluoropyridin-5-yl]-3-fluorophenyl]-2-oxooxazolidin-5-ylmethyl]acetamide,
N-[5(S)-3-[4-[4-(1-aminocyclopropan-1-yl)phenyl]-3,5-difluorophenyl]-2-oxooxazolidin-5-ylmethyl]acetamide,
N-[5(S)-3-[4-[2-(1-aminocyclopropan-1-yl)pyrimidin-5-yl]-3-fluorophenyl]-2-oxooxazolidin-5-ylmethyl]acetamide,
1-[5(R)-3-[4-[2-(1-aminocyclopropan-1-yl)pyridin-5-yl]-3,5-difluorophenyl]-2-oxooxazolidin-5-ylmethyl]-4-methyl-1,2,3-triazole,
1-[5(R)-3-[4-[2-(1-aminocyclopropan-1-yl)-3-fluoropyridin-5-yl]-3,5-difluorophenyl]-2-oxooxazolidin-5-ylmethyl]-4-methyl-1,2,3-triazole,
1-[5(R)-3-[4-[2-(1-aminocyclopropan-1-yl)pyridin-5-yl]-3,5-difluorophenyl]-2-oxooxazolidin-5-ylmethyl]-1,2,3-triazole, or
its enantiomer, diastereomer, or pharmaceutically acceptable salt, hydrate or prodrug thereof.

14. A pharmaceutical composition comprised of a compound in accordance with claim 1 in combination with a pharmaceutically acceptable carrier and optionally a in combination with a vitamin selected from the group consisting vitamin B2, vitamin B6, vitamin B12 and folic acid.

15. A method of treating or preventing a bacterial infection in a mammalian patient in need thereof, comprising administering to said patient an effective amount of a compound of claim 1.

16. A method of treating or preventing bacterial infection or an oxazolidinone-associated side effect by administering an effective amount of a compound of formula I of claim 1 and an effective amount of one or more of a vitamin selected from the group consisting of vitamin B2, vitamin B6, vitamin B12 and folic acid to a patient in need thereof.

17. A method according to claim 16 for treating or preventing oxazolidinone-associated normocyctic anemia, peripheral sensory neuropathy, sideroblastic anemia, peripheral sensory neuropathy, optic neuropathy, seizures, thrombocytopenia, cheilosis, hypo-regenerative anemia, megaloblastic anemia and seborrheic dermatitis by administering an effective amount of vitamin B2 to a patient in need thereof.
